SEQUENCE LISTING (8 sequences)

SEQ ID NO: 1 - DNA, 13 bp, Artificial sequence
  phosphorylated Bst XI adaptor
tcagttactcagg

SEQ ID NO: 2 - DNA, 17 bp, Artificial sequence
  phosphorylated Bst XI adaptor
cctgagtaactgacaca

SEQ ID NO: 3 - DNA, 20 bp, Artificial sequence
  PCR primer used for recovery of proviral inserts
cctcactccttctctagctc

SEQ ID NO: 4 - DNA, 23 bp, Artificial sequence
  PCR primer used for recovery of proviral inserts
aacaaattggactaatcgatacg

SEQ ID NO: 5 - DNA, 2639 bp, Homo sapiens
  cDNA sequence of GKLF
tcgaggcgaccgcgacagtggtgggggacgctgctgagtggaagagagcgcagcccggccaccggacctacttactcgccttgctgattgtctatttttgcgtttacaacttttctaagaacttttgtatacaaaggaactttttaaaaaagacgcttccaagttatatttaatccaaagaagaaggatctcggccaatttggggttttgggttttggcttcgtttcttctcttcgttgactttggggttcaggtgccccagctgcttcgggctgccgaggaccttctgggcccccacattaatgaggcagccacctggcgagtctgacatggctgtcagcgacgcgctgctcccatctttctccacgttcgcgtctggcccggcgggaagggagaagacactgcgtcaagcaggtgccccgaataaccgctggcgggaggagctctcccacatgaagcgacttcccccagtgcttcccggccgcccctatgacctggcggcggcgaccgtggccacagacctggagagcggcggagccggtgcggcttgcggcggtagcaacctggcgcccctacctcggagagagaccgaggagttcaacgatctcctggacctggactttattctctccaattcgctgacccatcctccggagtcagtggccgccaccgtgtcctcgtcagcgtcagcctcctcttcgtcgtcgccgtcgagcagcggccctgccagcgcgccctccacctgcagcttcacctatccgatccgggccgggaacgacccgggcgtggcgccgggcggcacgggcggaggcctcctctatggcagggagtccgctccccctccgacggctcccttcaacctggcggacatcaacgacgtgagcccctcgggcggcttcgtggccgagctcctgcggccagaattggacccggtgtacattccgccgcagcagccgcagccgccaggtggcgggctgatgggcaagttcgtgctgaaggcgtcgctgagcgcccctggcagcgagtacggcagcccgtcggtcatcagcgtcagcaaaggcagccctgacggcagccacccggtggtggtggcgccctacaacggcgggccgccgcgcacgtgccccaagatcaagcaggaggcggtctcttcgtgcacccacttgggcgctggaccccctctcagcaatggccaccggccggctgcacacgacttccccctggggcggcagctccccagcaggactaccccgaccctgggtcttgaggaagtgctgagcagcagggactgtcaccctgccctgccgcttcctcccggcttccatccccacccggggcccaattacccatccttcctgcccgatcagatgcagccgcaagtcccgccgctccattaccaagagctcatgccacccggttcctgcatgccagaggagcccaagccaaagaggggaagacgatcgtggccccggaaaaggaccgccacccacacttgtgattacgcgggctgcggcaaaacctacacaaagagttcccatctcaaggcacacctgcgaacccacacaggtgagaaaccttaccactgtgactgggacggctgtggatggaaattcgcccgctcagatgaactgaccaggcactaccgtaaacacacggggcaccgcccgttccagtgccaaaaatgcgaccgagcattttccaggtcggaccacctcgccttacacatgaagaggcatttttaaatcccagacagtggatatgacccacactgccagaagagaattcagtattttttacttttcacactgtcttcccgatgagggaaggagcccagccagaaagcactacaatcatggtcaagttcccaactgagtcatcttgtgagtggataatcaggaaaaatgaggaatccaaaagacaaaaatcaaagaacagatggggtctgtgactggatcttctatcattccaattctaaatccgacttgaatattcctggacttacaaaatgccaagggggtgactggaagttgtggatatcagggtataaattatatccgtgagttgggggagggaagaccagaattcccttgaattgtgtattgatgcaatataagcataaaagatcaccttgtattctctttaccttctaaaagccattattatgatgttagaagaagaggaagaaattcaggtacagaaaacatgtttaaatagcctaaatgatggtgcttggtgagtcttggttctaaaggtaccaaacaaggaagccaaagttttcaaactgctgcatactttgacaaggaaaatctatatttgtcttccgatcaacatttatgacctaagtcaggtaatatacctggtttacttctttagcatttttatgcagacagtctgttatgcactgtggtttcagatgtgcaataatttgtacaatggtttattcccaagtatgccttaagcagaacaaatgtgtttttctatatagttccttgccttaataaatatgtaatataaatttaagcaaacgtctattttgtatatttgtaaactacaaagtaaaatgaacattttgtggagtttgtattttgcatactcaaggtgagaattaagttttaaataaacctataatattttatctg

SEQ ID NO: 6 - protein, 470 aa, Homo sapiens
  amino acid sequence of GKLF protein
MAVSDALLPSFSTFASGPAGREKTLRQAGAPNNRWREELSHMKRLPPVLPGRPYDLAAATVATDLESGGAGAACGGSNLAPLPRRETEEFNDLLDLDFILSNSLTHPPESVAATVSSSASASSSSSPSSSGPASAPSTCSFTYPIRAGNDPGVAPGGTGGGLLYGRESAPPPTAPFNLADINDVSPSGGFVAELLRPELDPVYIPPQQPQPPGGGLMGKFVLKASLSAPGSEYGSPSVISVSKGSPDGSHPVVVAPYNGGPPRTCPKIKQEAVSSCTHLGAGPPLSNGHRPAAHDFPLGRQLPSRTTPTLGLEEVLSSRDCHPALPLPPGFHPHPGPNYPSFLPDQMQPQVPPLHYQELMPPGSCMPEEPKPKRGRRSWPRKRTATHTCDYAGCGKTYTKSSHLKAHLRTHTGEKPYHCDWDGCGWKFARSDELTRHYRKHTGHRPFQCQKCDRAFSRSDHLALHMKRHF

SEQ ID NO: 7 - RNA, 6 nt, Artificial sequence
  polyadenylation signal
aauaaa

SEQ ID NO: 8 - DNA, 7 bp, Artificial sequence
  minimal essential binding site for GKLF protein
rrggygy

(19) United States
(12) Patent Application Publication
(10) Pub. No.: US 2003/0138799 A1
(43) Pub. Date: Jul. 24, 2003

(54) Oncogene identification by transformation of RK3E cells and uses thereof

(76) Inventors: J. Michael Ruppert, Birmingham, AL (US); Jeffrey Allen Engler, Birmingham, AL (US)

Correspondence Address: Dr. Benjamin Adler, Adler & Associates, 8011 Candle Lane, Houston, TX, 77071, US

(21) Appl. No.: 10/194,527

(22) Filed: Jul. 12, 2002

Related U.S. Application Data
(63) Continuation-in-part of application No. 09/572,224, filed on May 17, 2000.
(60) Provisional application No. 60/134,936, filed on May 19, 1999.

(51) Int. Cl. C12Q 1/68; G01N 33/574; C12N 15/85

(52) U.S. Cl. 435/6; 435/7.23; 435/455

(57) ABSTRACT

The present invention is directed to methods of identifying new carcinoma oncogenes or analyzing functions of known carcinoma oncogenes by transformation of RK3E cells. Also provided are methods of identifying oncogene-specificity of known drugs or screening for new drugs that inhibit oncogenes activated in carcinoma by utilizing RK3E cells. Further provided are methods of identifying alterations in cellular enzyme, protein, or mRNA levels or activities by utilizing RK3E and oncogene-transformed derivatives. Still further provided are a novel oncogene GKLF with cDNA sequence and amino acid sequence for the protein, and applications of such gene/protein in medical diagnosis and treatment.

### CROSS-REFERENCE TO RELATED APPLICATION

This non-provisional patent application is a continuation in part of non-provisional application, U.S. Ser. No. 09/572,224, filed May 17, 2000 which claims benefit of provisional patent application U.S. Serial No. 60/134,936, filed May 19, 1999, now abandoned.

### FEDERAL FUNDING LEGEND

This invention was produced in part using funds obtained through NIH grant R29CA65686-05. Consequently, the federal government has certain rights in this invention.

### BACKGROUND OF THE INVENTION

1. Field of the Invention

The present invention relates generally to the molecular oncology. More specifically, the present invention relates to oncogene identification by transformation of RK3E cells and uses thereof. Even more specifically, the present invention relates to the newly identified oncogene Gut-Enriched Krüppel-Like Factor/Epithelial Zinc Finger (GKLF) a n d applications of such gene in medical diagnosis and treatment.

2. Description of the Related Art

Cellular oncogenes have been isolated by characterization of transforming retroviruses from animal tumors, by examination of the breakpoints resulting from chromosomal translocation, by expression cloning of tumor DNA molecules using mesenchymal cells such as NIH3T3, and by other methods (1-5). Several human tumor-types exhibit loss-of-function mutations in a tumor suppressor gene that lead to activation of a specific oncogene in a large proportion of tumors. For example, c-MYC expression is regulated by the APC colorectal tumor suppressor, expression of GLI is activated by loss-of-function of PTC in human basal cell carcinoma and i n animal models, E2F is activated by loss-of-function of the retinoblastoma susceptibility protein p105^{Rb}, and RAS GTPase activity is regulated by the familial neurofibromatosis gene NF1 (6-12). The comparative genomic hybridization assay and related methods have shown that numerous uncharacterized loci in tumors undergo gene amplification (13). These observations, and the infrequent genetic alteration of known oncogenies in certain tumor-types, suggest that novel transforming oncogenes remain to be identified.

One limitation to the isolation of oncogenes has been the paucity of in vitro assays for functional expression cloning, as several oncogenes are known to exhibit cell-type specificity. For example, GLI, BCR-ABL, NOTCH1/TAN1, and the G protein GIP2 have been found to transform immortalized rat cells (14-18), but not NIH3T3 or other cells, demonstrating the potential utility of alternate assays for oncogene expression cloning. While most studies have used NIH3T3 or other mesenchymal cells as host for analysis of oncogenes relevant to carcinoma, the potential utility of a host cell with epithelial characteristics has been discussed (2).

A consistent feature of human tumors is inactivation of the G1-phase cell-cycle regulatory pathway that includes p105^{Rb} (19-22). Loss-of-function mutations affect p105^{Rb} or the cyclin dependent kinase inhibitors, or gain-of-function mutations occur in cyclin-dependent kinases or associated cyclins. Such alterations are rate-limiting for tumor formation in vivo, since inheritance of these defects predisposes to retinoblastoma, cutaneous malignant melanoma, and other tumors. During viral infection of normal cells, disruption of the same pathway is critical for successful induction of the cellular DNA replicative machinery to support viral replication. Therefore, viruses express proteins such as adenovirus E1A that affect cell cycle progression through direct interaction with cell cycle regulators including p105^{Rb}, p27^{Kipl}, and others (23-26).

Thus, the prior art is deficient in methods of identifying carcinoma oncogenes by utilizing RK3E cells. The present invention fulfills this long-standing need and desire in the art.

### SUMMARY OF THE INVENTION

RK3E cells, immortalized by E1A, were previously utilized t o demonstrate the transforming activity of GLI (17). The present invention demonstrates that these cells exhibit multiple features of epithelia and detect known and novel transforming activities in tumor cell lines. The epithelial features of the cells and/or the mechanism of immortalization may explain the surprising sensitivity and specificity of the assay compared with previous expression cloning approaches (27). Three of the four genes known to transform RK3E cells are activated by genetic alterations in carcinomas, and of these genes only RAS exhibits transforming activity in the commonly-used host NIH3T3. GKLF is hereby identified as an oncogene expressed in the differentiating compartment of epithelium and misexpressed in dysplastic epithelium. GKLF may regulate the rate of differentiation and maturation and the overall cellular transit time through epithelium. The function al similarities shared with other oncogenes including GLI or c-MYC identify GKLF as an attractive candidate gene relevant to tumor pathogenesis.

The present invention describes an RK3E assay for oncogene identification and oncogene-specificity drug screening. As a result of the assay, GKLF is identified as an oncogene. The present invention further describes that this oncogene can be used in medical evaluation and treatment.

In one embodiment of the present invention, there is provided a method of detecting transforming activities of a carcinoma oncogene, comprising the steps of transforming epithelioid cells with the oncogene and then detecting morphological transformation, wherein the presence of transformed cell lines indicates that the oncogene has transforming activities. Preferably, the epithelioid cells are RK3E cells.

In another embodiment of the present invention, there is provided a method of identifying oncogenicity of a gene, comprising the steps of transforming epithelioid cells with the gene; detecting transformed cell lines and measuring tumorigenicity of said transformed cell lines by injecting the transformed cell lines into an animal, wherein induction of tumors in the animal indicates that the gene is a oncogene. Preferably, the epithelioid cells are RK3E cells.

In still another embodiment of the present invention, there is provided a method of identifying oncogene-specificity of a known drug, comprising the steps of transforming epithelioid cells with the oncogene; detecting transformed cell lines and contacting the transformed cell lines with the drug, wherein if the drug inhibits proliferation or survival of the transformed cell lines, the drug is specific for the oncogene. Preferably, the epithelioid cells are RK3E cells.

In still yet another embodiment of the present invention, there is provided a method of screening for a drug functioning as an inhibitor of an oncogene, comprising the steps of transforming epithelioid cells with the oncogene; contacting the cells with the test drug and detecting transformed cell lines, wherein absence of transformation or reduced transformation compared to the result obtained without the drug contact indicates that the test drug is an inhibitor of the oncogene. Preferably, the epithelioid cells are RK3E cells.

The present invention is further directed to a method of screening for alterations in enzyme activity, protein expression, or mRNA expression in association with an oncogene, comprising the steps of: transforming epithelioid cells with said oncogene; and measuring said enzyme, protein or mRNA levels or activities; wherein alterations in transformed cell lines vs. in non-transformed cell lines indicate that the oncogene regulates the enzyme activity, protein expression, or mRNA expression.

Still further provided is a method of treating an individual having a carcinoma by administering a drug to the individual, wherein the drug inhibits the expression/activity of GKLF.

In yet another embodiment of the present invention, there is provided a method of monitoring a treatment thereby evaluating effectiveness of the treatment in an individual, comprising the step of detecting the expression levels of GKLF in the individual prior to, during (and post said treatment, wherein decreases of GKLF expression levels indicate effective response of the individual to the treatment. By doing so, the treatment is monitored and the effectiveness of the treatment is evaluated in the individual.

The present invention further provides a monoclonal antibody directed against GKLF protein, wherein the antibody is an IgG₁ antibody raised against bacterially-expressed GKLF. Such antibody can be used t o monitor a treatment, further evaluate effectiveness of the treatment in a n individual.

Still further provided in the present invention is a kit for monitoring a treatment thereby evaluating effectiveness of the treatment in an individual, comprising the monoclonal antibody disclosed herein and a suitable carrier.

Yet furthermore, the present invention provides a DNA fragment encoding a Gut-Enriched Krüppel-Like Factor/Epithelial Zinc Finger (GKLF) protein selected from the group consisting of: (a) isolated DNA which encodes a GKLF protein; (b) isolated DNA which hybridizes to isolated DNA of (a) and which encodes a GKLF protein; and (c) isolated DNA differing from the isolated DNAs of (a) and (b) in codon sequence due to the degeneracy of the genetic code, and which encodes a GKLF protein. Preferably, the DNA has the sequence shown in SEQ ID No: 5; and the GKLF protein has the amino acid sequence shown in SEQ ID No: 6.

In yet another embodiment of the present invention, there is provided a vector capable of expressing the DNA fragment disclosed herein adapted for expression in a recombinant cell and regulatory elements necessary for expression of the DNA fragment in the cell; and a host cell transfected with such vector. Preferably, the host cell is selected from group consisting of bacterial cells, mammalian cells, plant cells and insect cells. An example of bacterial cell is E. coli.

In still yet another embodiment of the present invention, there is provided an isolated and purified GKLF protein coded for by DNA fragment selected from the group consisting of: (a) isolated DNA which encodes a GKLF protein; (b) isolated DNA which hybridizes to isolated DNA of (a) and which encodes a GKLF protein; and (c) isolated DNA differing from the isolated DNAs of (a) and (b) in codon sequence due to the degeneracy of the genetic code, and which encodes a GKLF protein. Preferably, the GKLF protein has the amino acid sequence shown in SEQ ID No: 6.

In still yet another embodiment of the present invention, there is provided a method of identifying the prognosis of an individual thereby allowing selection of a more effective, less invasive or a less toxic therapeutic alternative to individual patients having a breast tumor, comprising the step of examining the expression of KLF4 in said breast tumor.

In still yet another embodiment of the present invention, there is provided a cell line generating a monoclonal antibody directed against KLF4 protein. A representative example of such an antibody is the monoclonal antibody designated IE5/IE2.

Other and further aspects, features, and advantages of the present invention will be apparent from the following description of the presently preferred embodiments of the invention. These embodiments are given for the purpose of disclosure.

### BRIEF DESCRIPTION OF THE DRAWINGS

So that the matter in which the above-recited features, advantages and objects of the invention, as well as others which will become clear, are attained and can be understood in detail, more particular descriptions of the invention briefly summarized above may be had by reference to certain embodiments thereof which are illustrated in the appended drawings. These drawings form a part of the specification. It is to be noted, however, that the appended drawings illustrate preferred embodiments of the invention and therefore are not to be considered limiting in their scope.

FIG. 1 shows that RK3E exhibit characteristics of epithelial cells. FIG. 1A: Confluent RK3E cells in a culture dish were fixed and stained with uranyl acetate and lead citrate, and ultra-thin sections were examined using a Hitachi 7000 transmission electron microscope. The upper surface was exposed to growth media, and the lower surface was adherent. Electron dense aggregates typical of adherens junctions (arrows) and desmosomes (circled) are shown. Bars, 3.2 μm (top panel) or 1.3 μm (bottom panel). FIG. 1B: Northern blot analysis of RK3E cells (lane 1) and REF52 fibroblasts (lane 2). The filter was hybridized sequentially to a desmoplakin probe (upper) and then to β-tubulin (lower). FIG. 1C: Vimentin expression by immunocytochemistry in RK3E (top) and REF52 (bottom) cells. Bars, 100 μm.

FIG. 2 shows expression cloning of c-MYC and GKLF. FIG. 2A: Identification of human cDNAs present in transformed RK3E cell lines SQC1-SQC13 (derived using a squamous cell carcinoma library, lanes 1 and 3-14) and BR1 (derived using a breast carcinoma library, lane 15). The polymerase chain reaction (PCR) was used in combination with vector-derived primers and cell line genomic DNA. RK3E genomic DNA served as a negative control template (lane 2). No cDNA was retrieved from cell line SQC3 (lane 4). All foci identified in the screen are represented. Molecular weight markers are indicated on the left in kilobase-pairs. FIG. 2B: Reconstitution of transforming activity by cloned PCR products. cDNAs were cloned into a retroviral expression plasmid, packaged into virus using BOSC23 cells, and applied to RK3E cells. Foci were fixed and stained at 3-4 weeks. Vector: pCTV3K; Control: pCTV3K-SQC1; c-MYC: pCTV3K-BR1; GKLF: pCTV3K-SQC7. FIG. 2C: Morphology of foci and cloned cell lines. Top to bottom: first panel, low power phase contrast view of adjacent foci in a dish transduced with retrovirus encoding GKLF; bar, 900 μm. Second through fourth panels: high power phase contrast view; bar, 230 μm; second panel, RK3E cells at subconfluence; third panel, GKLF-transformed RK3E cells; fourth panel, c-MYC-transformed RK3E cells.

FIG. 3 shows Northern blot analysis of c-MYC and GKLF expression. 25 μg of total RNA was loaded for each sample. FIG. 3A: Analysis of transgene expression in RK3E cells and derivative cell lines transformed by the indicated oncogene. Lane 1: RK3E cells in exponential growth phase; lane 2: RK3E incubated at confluence for five days. Ethidium bromide-stained RNA is shown below after transfer to the filter. FIG. 3B: Endogenous GKLF (3.0 kb) or c-MYC (2.3 kb) expression in tumor cell lines. Lanes 1-3: breast cancer lines; lanes 4-6: squamous cell carcinoma lines. FIG. 3C: Analysis of gene expression in laryngeal squamous cell carcinoma. Lane 1: SCC25 cell line; lanes 3-6, 9, 12: primary tumors; lanes 7, 8, 10 and 11: metastatic tumors. Lanes 3-12 correspond to case numbers 5, 8, 18-20, 6, and 21-24, respectively (see Table 4). RK3E-RAS cell RNA served as a negative control (lane 2), while hybridization to β-tubulin served as a control for loading.

FIG. 4 shows Southern blot analysis of cell line- and tumor-derived genomic DNA. 5 μg of DNA was digested with EcoRI and separated by gel electrophoresis. The filters were hybridized sequentially to GKLF, c-MYC, and β-tubulin probes. Asterisks indicate samples with increased apparent copy number of c-MYC. Molecular weight markers are indicated on the right. NL, normal human lymphocyte DNA. FIG. 4A: Oropharyngeal squamous cell carcinoma. Cell lines (lanes 2-4) and tumors (lanes 5-15) are shown. FIG. 4B: Breast carcinoma. Cell lines (lanes 2-5) and tumors (lanes 6-14) are shown.

FIGS. 5A/5B shows in situ hybridization analysis of GKLF. Paraffin-embedded (A-L) or fresh-frozen (M-O) tissues were analyzed using antisense (GKLF-AS) or sense (GKLF-S) ³⁵S-labelled RNA probes. Each image (A-O) is 650 μm×530 μm. Sections were stained with Hematoxylin and Eosin (H&E). Case 1, A-C: uninvolved epithelium in a patient with primary laryngeal squamous cell carcinoma; D-F: adjacent dysplastic epithelium within the same tissue block. Case 2, G-I: uninvolved epithelium; J-L: adjacent primary tumor nests within stroma in the same tissue block; asterisk indicates a salivary gland and ducts. Case 3, M-O: metastatic laryngeal squamous cell carcinoma infiltrating a lymph node; asterisk indicates lymphocytes.

FIG. 6 shows in situ hybridization analysis of GKLF mRNA in carcinoma of the breast. Two distinct cases were analyzed by applying an antisense (GKLF-AS) [³⁵S]-labeled RNA probe to sections of parraffin-embedded (A) or fresh-frozen (B) surgical material. Brightfield (left) and darkfield (right) views are shown. Sections were stained with hematoxylin and eosin (H&E). Two areas of the same slide are shown in FIG. 6A, with uninvolved (i.e., morphologically normal) breast epithelium (upper plate) adjacent to an area (lower plate) containing DCIS (arrowheads) and additional uninvolved tissue (arrows). FIG. 6B shows invasive ductal carcinoma admixed with cords of stroma. Scale bars=160 μm.

FIG. 7 shows GKLF mRNA expression in normal and neoplastic breast tissue. The data in Table 5 was analyzed using a paired t-test. Sample size (N), statistical significance (p), and standard error of the mean are indicated for each comparison. Uninv, uninvolved ducts; DCIS, ductal carcinoma in situ; IDC, invasive ductal carcinoma.

FIG. 8 shows immunostaining of human tissues with αGKLF monoclonal antibody. Each panel (FIG. 8A-C) illustrates adjacent areas of a tissue section. FIG. 8A, uninvolved oral epithelium (left) and invasive oral squamous cell carcinoma (right). Arrowheads indicate the basal cell layer, while arrows indicate invasive carcinoma. Staining of tumor cells and of superficial epithelial cells is indicated by a brown precipitate. FIG. 8B, a section of small bowel illustrating increased staining of superficial epithelium (left) compared to cells deeper within crypts (right). FIG. 8C, a case of colorectal carcinoma, with increased staining of uninvolved superficial mucosa (left) compared to adjacent tumor cells (right). Scale bar for C (left panel)=45 μm; other scale bars=140 μm.

FIG. 9 shows immunostaining of breast tissue with αGKLF. FIG. 9A shows a tissue section containing uninvolved epithelium (left, arrowheads) adjacent to invasive carcinoma (right); FIG. 9B shows a different case showing invasive carcinoma cells with a mixed nuclear and cytoplasmic staining pattern. FIG. 9C shows a tissue section containing an uninvolved duct (left panel) adjacent to both DCIS (right panel, arrows) and invasive carcinoma (right panel, arrowheads). Scale bars: A=120 μm; B=30 μm; C=60 μm.

FIG. 10 shows staining of uninvolved (FIG. 10A) and neoplastic (FIG. 10B) breast tissue by αGKLF. The data in Table 6 were analyzed using a paired t-test. Sample size (N), statistical significance (p), and standard error of the mean are indicated for each comparison. Uninv, uninvolved ducts; DCIS, ductal carcinoma in situ; IDC, invasive ductal carcinoma.

FIG. 11 shows Northern blot analysis of GKLF expression i n human breast tumor cell lines. Total RNA from the indicated cell lines was analyzed. Lane 1, finite-lifespan HMECs; lane 2, benzo(a)pyrene-treated, immortalized HMECs; lanes 3-10, breast carcinoma-derived cell lines; lane 11, SCC15, a human oral squamous cell carcinoma-derived cell line; lane 12, a RAS-transformed rat cell line. The filter was stripped and hybridized to a β-tubulin probe.

FIG. 12 shows survival rates of invasive breast cancer patients according to GKLF staining patterns in the cytoplasm and nucleus (includes small tumors only).

FIG. 13 shows survival rates of invasive breast cancer patients according to GKLF staining patterns in the cytoplasm and nucleus (using the median immunoscore as the cut off). FIG. 13A shows the staining pattern of low cytoplasmic GKLF/high nuclear GKLF vs. all other profiles. FIG. 13B shows the staining pattern of low cytoplasmic GKLF/high nuclear GKLF vs. high cytoplasmic GKLF/low nuclear GKLF.

### DETAILED DESCRIPTION OF THE INVENTION

The function of several known oncogenes is restricted to specific host cells in vitro, suggesting that new genes may be identified by using alternate hosts. RK3E cells exhibit characteristics of epithelia and are susceptible to transformation by the G protein RAS and the zinc finger protein GLI. Expression cloning identified the major transforming activities in squamous cell carcinoma cell lines as c-MYC and the zinc fingers protein Gut-Enriclhed Krüppel-Like Factor/Epithelial Zinc Finger (GKLF). In oral squamous epithelium, GKLF expression was detected in the upper, differentiating cell layers. In dysplastic epithelium expression was prominently increased and was detected diffusely throughout the entire epithelium, indicating that GKLF is misexpressed in the basal compartment early during tumor progression. The results demonstrate transformation of epithelioid cells to be a sensitive and specific assay for oncogenes activated during tumorigenesis in vivo, and identify GKLF as an oncogene that may function as a regulator of proliferation or differentiation in epithelia.

The present study further utilized in situ hybridization, Northern blot analysis, and immunohistochemistry to detect GKLF at various stages of tumor progression in the breast, prostate, and colon. Overall, expression of GKLF mRNA was detected by in situ hybridization in 21 of 31 cases (68%) of carcinoma of the breast. Low-level expression of GKLF mRNA was observed in morphologically normal (uninvolved) breast epithelium adjacent to tumor cells. Increased expression was observed in neoplastic cells compared with adjacent uninvolved epithelium for 14 of 19 cases examined (74%). Ductal carcinoma in situ exhibited similar expression as invasive carcinoma, suggesting that GKLF is activated prior to invasion through the basement membrane. Expression as determined by Northern blot was increased in most breast tumor cell lines and in immortalized human mammary epithelial cells (HMECs) when these were compared with finite-lifespan human mammary epithelial cells. Alteration of GKLF expression was confirmed by use of a novel monoclonal antibody that detected the protein in normal and neoplastic tissues in a distribution consistent with localization of the mRNA. In contrast to most breast tumors, expression of GKLF in tumor cells of colorectal or prostatic carcinomas was reduced or unaltered compared with normal epithelium. The results demonstrate that GKLF expression in epithelial compartments is altered in a tissue-type specific fashion during tumor progression, and suggest that increased expression of GKLF mRNA and protein may contribute to the malignant phenotype of breast tumors.

The present invention demonstrates that transformation of RK3E represents a significant improvement over NIH3T3 transformation that are often used for oncogene analysis in vitro. RK3E assay can detect carcinoma oncogenes with sensitivity. Of the five genes disclosed in the present invention that function in RK3E cells, i.e., RAS, GKLF, c-MYC, GLI and SCC7, only RAS transforms NIH3T3 cells. RK3E assay can also detect new oncogenes with specificity, i.e., without artifacts from truncation or rearrangement. In addition, Rk3E cells are diploid and genetically stable.

In one embodiment of the present invention, there is provided a method of detecting transforming activities of a carcinoma oncogene, comprising the steps of transforming epithelioid cells with the oncogene and then detecting morphological transformation, wherein the presence of transformed cell lines indicates that the oncogene has transforming activities. Preferably, the epithelioid cells are RK3E cells. Representative examples of the oncogene include, but are not limited to, RAS, GKLF, c-MYC, GLI. Still preferably, the disclosed method detects protein coding region of the oncogene without truncation or rearrangement.

In another embodiment of the present invention, there is provided a method of identifying oncogenicity of a gene, comprising the steps of transforming epithelioid cells with the gene; detecting transformed cell lines and measuring tumorigenicity of said transformed cell lines by injecting the transformed cell lines into an animal, wherein induction of tumors in the animal indicates that the gene is an oncogene. Preferably, the epithelioid cells are RK3E cells.

In still another embodiment of the present invention, there is provided a method of identifying oncogene-specificity of a known drug, comprising the steps of transforming epithelioid cells with the oncogene; detecting transformed cell lines and contacting the transformed cell lines with the drug, wherein if the drug inhibits proliferation or survival of the transformed cell lines, the drug is specific for the oncogene. Preferably, the epithelioid cells are RK3E cells. Still preferably, the oncogene is activated in carcinoma and representative examples of oncogenes include RAS, GKLF, c-MYC, and GLI.

In still yet another embodiment of the present invention, there is provided a method of screening for a drug functioning as an inhibitor of an oncogene, comprising the steps of transforming epithelioid cells with the oncogene; contacting the cells with the test drug and detecting transformed cell lines, wherein absence of transformation or reduced transformation compared to the result obtained without the drug contact indicates the test drug is an inhibitor of the oncogene. Preferably, the epithelioid cells are RK3E cells. Still preferably, the oncogene is activated in carcinoma and examples of the oncogene include RAS, GKLF, c-MYC, GLI.

In still yet another embodiment of the present invention, there is provided a method for identification of oncogene-specific alterations in activity of signal transduction molecules or in the expression of cellular mRNAs, comprising the steps of transforming epithelioid cells with the oncogene; measuring enzyme activity or mRNA expression levels, wherein specific alteration of these parameters indicates the enzyme or mRNA is likely to be regulated by the oncogene. Preferably, the epithelioid cells are RK3E cells. Still preferably, the oncogene is activated in carcinoma and examples of the oncogene include, but are not limited to, RAS, GKLF, c-MYC, GLI.

The present invention is further directed to a method of screening for alterations in enzyme activity, protein expression, or mRNA expression in association with an oncogene, comprising the steps of: transforming epithelioid cells with said oncogene; and measuring said enzyme, protein or mRNA levels or activities; wherein alterations in transformed cell lines vs. in non-transformed cell lines indicate that the oncogene regulates the enzyme activity, protein expression, or mRNA expression. Preferably, the epithelioid cells are RK3E cells and the oncogene is a carcinoma oncogene. Representative oncogene include RAS, GKLF, c-MYC and GLI.

Still further provided is a method of treating an individual having a carcinoma by administering a drug to the individual, wherein the drug inhibits the expression/activity of GKLF. Representative examples of carcinoma include breast carcinoma and oral squamous cell carcinoma.

In yet another embodiment of the present invention, there is provided a method of monitoring a treatment thereby evaluating effectiveness of the treatment in an individual, comprising the step of detecting the expression levels of GKLF in the individual prior to, during and post said treatment, wherein decreases of GKLF expression levels indicate effective response of the individual to the treatment. By doing so, the treatment is monitored and the effectiveness of the treatment is evaluated in the individual. The treatments can be drug administration, radiation therapy, gene therapy, or chemotherapy. The individual may suffer from a carcinoma such as breast carcinoma and oral squamous cell carcinoma.

The present invention further provides a monoclonal antibody directed against GKLF protein, wherein the antibody is an IgG₁ antibody raised against bacterially-expressed GKLF. Such antibody can be used to monitor a treatment, further evaluate effectiveness of the treatment in an individual. Specifically, the monoclonal antibody detects the localization and level of GKLF protein, and wherein decreases of GKLF protein level indicate effective response of the individual to the treatment.

Still further provided in the present invention is a kit for monitoring a treatment thereby evaluating effectiveness of the treatment in an individual, comprising the monoclonal antibody disclosed herein and a suitable carrier.

Yet furthermore, the present invention provides a DNA fragment encoding a Gut-Enriched Krüppel-Like Factor/Epithelial Zinc Finger (GKLF) protein selected from the group consisting of: (a) isolated DNA which encodes a GKLF protein; (b) isolated DNA which hybridizes to isolated DNA of (a) and which encodes a GKLF protein; and (c) isolated DNA differing from the isolated DNAs of (a) and (b) in codon sequence due to the degeneracy of the genetic code, and which encodes a GKLF protein. Preferably, the DNA has the sequence shown in SEQ ID No: 5; and the GKLF protein has the amino acid sequence shown in SEQ ID No: 6.

In yet another embodiment of the present invention, there is provided a vector capable of expressing the DNA fragment disclosed herein adapted for expression in a recombinant cell and regulatory elements necessary for expression of the DNA fragment in the cell; and a host cell transfected with such vector. Preferably, the host cell is selected from group consisting of bacterial cells, mammalian cells, plant cells and insect cells. An example of bacterial cell is E. coli.

In still yet another embodiment of the present invention, there is provided an isolated and purified GKLF protein coded for by DNA fragment selected from the group consisting of: (a) isolated DNA which encodes a GKLF protein; (b) isolated DNA which hybridizes to isolated DNA of (a) and which encodes a GKLF protein; and (c) isolated DNA differing from the isolated DNAs of (a) and (b) in codon sequence due to the degeneracy of the genetic code, and which encodes a GKLF protein. Preferably, the GKLF protein has the amino acid sequence shown in SEQ ID No: 6.

The present invention is also directed to a method of identifying the prognosis of an individual thereby allowing selection of a more effective, less invasive or a less toxic therapeutic alternative to individual patients having a breast tumor, comprising the step of examining the expression of KLF4 in said breast tumor. Preferably, the expression is examined using a technique such as immunohistochemistry. In a preferred embodiment, the immunohistochemistry employs a monoclonal antibody directed against KLF4 protein. Generally, a predominantly cytosolic staining indicates a greater likelihood of survival of the individual or a greater likelihood of response to a specific therapy (e.g., local or loco-regional resection in surgery, chemotherapy agents, radiotherapy, or hormonal therapy). In constrast, a predominantly nuclear staining and a lower cytosolic staining indicates a lower likelihood of survival of the individual or a lower likelihood of response to a specific therapy (e.g., local or loco-regional resection in surgery, chemotherapy agents, radiotherapy, or hormonal therapy). This prognostic method may be particularly valuable when the tumor is smaller than about 2 cm.

The present invention is also directed to a cell line generating a monoclonal antibody directed against KLF4 protein. Preferably, the cell line generates a monoclonal antibody such as the antibody antibody designated IE5/IE2.

The following examples are given for the purpose of illustrating various embodiments of the invention and are not meant to limit the present invention in any fashion:

### EXAMPLE 1

Immunocytochemistry

Immunocytochemical assays were performed in the Immunopathology Laboratory at The University of Alabama at Birmingham. Antibodies to vimentin and desmin were from Dako (Carpenteria, Calif.). A cocktail of anti-cytokeratin included AE1/AE3 (Biogenics, San Ramon, Calif.), CAM5.2 (Becton Dickinson, San Jose, Calif.), and MAK-6 (Zymed, So. San Francisco, Calif.). Human tissue served as a positive control for each antibody. No signal was obtained in the absence of primary antibody.

### EXAMPLE 2

Construction of cDNA Libraries

Two cDNA libraries were constructed using the ZAP-Express™ cDNA synthesis kit (Stratagene, La Jolla, Calif.). A library was prepared from human squamous cell carcinoma cells derived from tumors of the oro-pharynx. Equal quantities of total mRNA from cell lines SCC15, SCC25, and FaDu (ATCC, Rockville, Md.) were pooled. Similarly, equal quantities of mRNA from the breast cancer cell lines MCF-7, ZR75-1, MDAMB-453, and T47D (ATCC) were pooled. For each pool, poly A+mRNA was selected by two cycles of oligo-dT cellulose affinity chromatography. 5 μg was reverse transcribed using an oligo-dT linker primer and MMLV reverse transcriptase. Double-stranded cDNA was synthesized using E. coli RNAase H and DNA polymerase I. cDNA was ligated to λZAP EXPRESS™ bacteriophage arms and packaged into virions. The λ titer and the frequency of non-recombinants was determined prior to amplification of the library on bacterial plates (Table 1). The frequency of non-recombinant clones was estimated to be less than 2% by complementation of β-gal activity (blue/white assay). Phage were converted to pBKCMV plasmids by autoexcision in bacteria. Insert sizes in randomly selected clones were determined at this step by gel electrophoresis of plasmid DNA digested with Sal I and Not I (Table 1). The pBKCMV plasmid libraries were amplified in soft agar at 4×10⁴ colony forming units per ml (27). After incubation at 37° C. for 15 hrs, bacterial cells within the agar bed were isolated by centrifugation, amplified for 3-4 doublings in culture, and plasmid DNA was purified using a Qiagen column (Qiagen, Inc., Chatsworth, Calif.).

TABLE 1
Assessment of cDNA libraries

| | | | | cDNA | Transduce | |
|---|---|---|---|---|---|---|
| | | cDNA siz | | clone | RK3E | Foci |
| Library | λ tite | (N, R)^{a} | Probe | transduced | cells | identi |
| Squamou | 8.9 × 10 | 1.69 | NT | ˜4 × 10⁶ | ˜1.2 × 10⁷ | 13 |
| cell ca. | | (10, | | | | |
| | | 13.60) | | | | |
| Breast ca | 7.4 × 10 | 1.64 | hBRF | ˜4 × 10⁶ | ˜1.2 × 10⁷ | 1 |
| | | (18, 02.7) | | | | |

^{a}indicates mean size of cDNAs in kilobase-pairs, the number of clones sized by gel electrophoresis (N), and the size range (R).
^{b}420,000 plaques were analyzed by hybridization to the 5′ end of the RNA polymerase III transcription factor hBRF cDNA.
NT, not tested.
^{c}The number of clones processed at each step of library construction was equal to or greater than 4 × 10⁶. The Bst XI adaptor strategy generates recombinant cDNA expression plasmids in an orientation-independent fashion, such that both sense and antisense vectors result.
^{d}The number of RK3E cells transduced was estimated as the product of the transduction frequency (20%), the number of dishes screened (20), and the number of cells per dish (3 × 10⁶).

To generate libraries in a retroviral expression vector, cDNA inserts were excised from 10 μg of plasmid using Sal I and Xho I. After treatment with Klenow and dNTPs and extraction with phenol, the DNA was ligated to 5′ phosphorylated Bst XI adaptors (5′-TCAGTTACTCAGG-3′ (SEQ ID No. 1) and 5′-CCTGAGTAACTGACACA-3′ (SEQ ID No. 2)) as described (27). After treatment with Not I, excess adaptors were removed by gel filtration, and the residual vector was converted to a 9.0 kb dimer using the Not I site and T4 DNA ligase. The cDNA was size fractionated by electrophoresis in Sea Plaque® agarose (FMC BioProducts, Rockland, Me.) and fragments 0.6-8.5 kb were isolated and ligated to the Bst XI- and alkaline phosphatase-treated MMLV retroviral vector pCTV1B (27). E. coli MC1061/p3 were transformed by electroporation and selected in soft agar as above.

### EXAMPLE 3

Retroviral Transduction

The libraries were analyzed in two transfection experiments performed on consecutive days. For each library, ten 10 cm. dishes of BOSC23 ecotropic packaging cells at 80%-90% confluence were transfected using 30 μg of plasmid DNA per dish (29). The transfection efficiency for these cells was ˜60%, as determined using a β-gal control plasmid. Viruses were collected in a volume of 9.0 mls/dish at 36-72 hours post-transfection, filtered, and the 9.0 mls was expressed into a 10 cm dish containing RK3E cells at ˜30% confluence. Polybrene was added to a final concentration of 10 μg/ml. After 15 hours, and every three days thereafter, the cells were fed with growth media (17). A total of 20 RK3E dishes were transduced for each library. A β-gal retroviral plasmid transduced at least 20-30% of RK3E cells in control dishes. For colony assays hygromycin was used at 100 μg/ml. Cell proliferation rates for transformed cell lines was measured by plating 2×10⁵ cells in duplicate and counting cells 96 hours later using a hemacytometer.

### EXAMPLE 4

Polymerase Chain Reaction (PCR) Recovery of Proviral Inserts

PCR reactions used 200 ng of cell line genomic DNA, 20 mM Tris-HCl (pH 8.8), 87 mM potassium acetate, 1.0 mM MgCl₂, 8% glycerol, 2% dimethylsulfoxide, 0.2 mM of each dNTP, 32 pmol of each primer (5′-CCTCACTCCTTCTCTAGCTC-3′ (SEQ ID No. 3); 5′-AACAAATTGGACTAATCGATACG-3′ (SEQ ID No. 4)) (27), 5 units of Taq polymerase (Gibco BRL, Gaithersburg, Md.), and 0.3 units of Pfu polymerase (Stratagene, La Jolla, Calif.) in a volume of 0.05 ml. Cycling profiles were: 95° C. for 1 min; then 95° C. for 10 s, 59° C. for 40 s, 68° C. for 8 min (35 cycles).

### EXAMPLE 5

RNA Extraction and Northern Blot Analysis

Tumor samples were obtained through the Tissue Procurement Facility of the UAB Comprehensive Cancer Center and the Southern Division of the Cooperative Human Tissue Network. Microdissection was used to isolate tissue composed of>70% tumor cells. Total RNA was isolated as described (59), then denatured and separated on a 1.5% formaldehyde agarose gel and transferred to nitrocellulose (Schleicher & Schuell, Keene, N.H.). Prehybridization was at 42° C. for 3 hours in 50% formamide, 4×SSC (SSC is 150 mM NaCl, 15 mM sodium citrate, pH 7.5), 0.1 M sodium phosphate (pH 6.8), 0.1% sodium pyrophosphate, 0.1% SDS, 5×Denhardt's and 25 μg/ml denatured salmon sperm DNA. Hybridization was at 42° C. for 16-20 hrs. The hybridization mixture contained 45% formamide, 4×SSC, 0.1 M sodium phosphate (pH 6.8), 0.075% sodium pyrophosphate, 0.1% SDS, 10% dextran sulfate and 100 μg/ml denatured salmon sperm DNA. Following hybridization, the filter was washed twice in 2×SSC, 0.1% SDS for 20 minutes at room temperature, then washed in 0.3×SSC, 0.3% SDS for 30 min at 59° C. (for detection of rat transcripts) or 65° C. For stripping of hybridized probes, the filter was placed in a solution of 2×SSC, 25 mM Tris-HCl (pH 7.5), 0.1% SDS at initial temperature of 95° C., and shaken for 10 min at room temperature.

### EXAMPLE 6

In Situ Hybridization

In situ hybridization was conducted as described (60), using sense and antisense ³⁵S-labelled riboprobes generated from a 301 base pair EcoRI fragment derived from the GKLF 3′ UTR positioned 40 bases from the stop codon. A GAPDH antisense probe corresponding to bases 366-680 (Accession M33197) was synthesized using a commercially available template (Ambion, Inc., Austin, Tex.). All results were obtained in duplicate. High stringency washes were in 0.1×SSC and 0.1% (v/v) 2-mercaptoethaniol at 58° C. for GKLF or 68° C. for GAPDH. Slides were coated with emulsion and exposed for 14 days.

### EXAMPLE 7

Nucleotide Sequencing

Automated sequence analysis was performed for the two independent GKLF isolates using vector-derived primers and sense or antisense primers spaced at 400 bp intervals within the inserts. The complete sequence was obtained for both clones, with one of the clones analyzed for both strands. Primer sequences are available upon request. GKLF sequence was submitted to GenBank (Accession AF105036). The cDNA and amino acid sequences of GKLF are listed in SEQ ID No. 5 and SEQ ID No. 6, respectively.

### EXAMPLE 8

RK3F Cells Have Characteristics of Epithelia

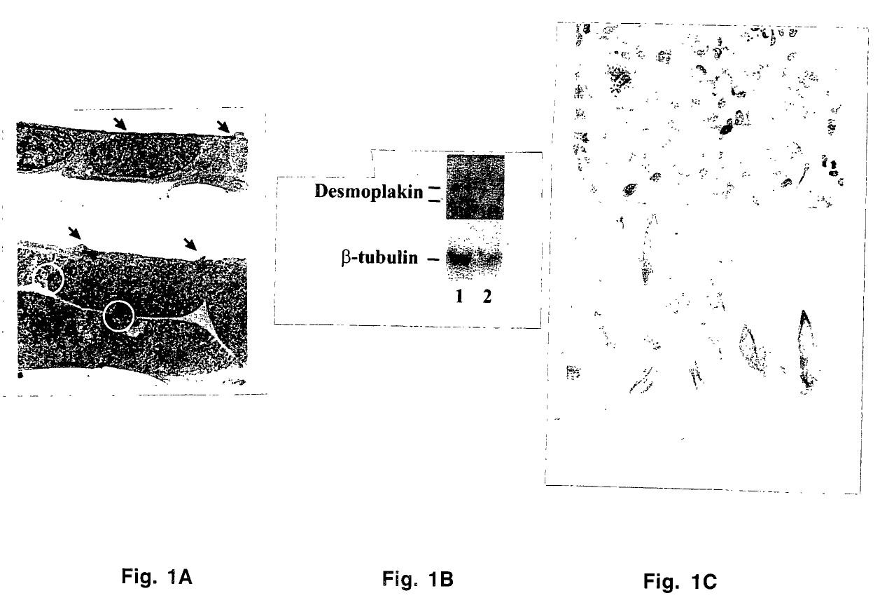

RK3E cells are a clone of primary rat kidney cells immortalized by transfection with adenovirus E1A in vitro (17). The cells exhibit morphological and molecular features that are epithelioid. They are contact-inhibited at confluence and are polarized with apical and basolateral surfaces and electron-dense intercellular junctions typical of adherens junctions and desmosomes (FIG. 1A). Northern blot analysis showed that RK3E cells, but not REF52 fibroblasts, expressed desmoplakin, at major component of desmosomes and an epithelial marker (FIG. 1B). By immunocytochemical staining, the mesenchymal marker vimentin was low or undetectable in RK3E cells but was strongly positive in REF52 cells (FIG. 1C). Neither line reacted strongly with anti-cytokeratin or anti-desmin antibodies. These results are consistent with the observation that E1A induces multiple epithelial characteristics without inducing cytokeratin expression (28).

Karyotype analysis revealed RK3E cells to be diploid with a slightly elongated chromosome 5 q as the only apparent abnormality (17). Importantly, RK3E cells can be transformed by functionally diverse oncogenes such as RAS and GLI. Four such transformed lines were each homogeneous for DNA content, as determined by fluorescence analysis of propidium iodide stained cells derived from RAS- (one line) or GLI- (three lines) induced foci, indicative of a relatively stable genetic constitution. These properties suggested that RK3E cells may serve as an in vitro model for identification and mechanistic analysis of gene products involved in the progression from normal epithelial tissue to malignancy.

### EXAMPLE 9

cDNA Library Construction

To identify transforming genes, mRNA from human squamous cell carcinoma- or breast tumor-derived cell lines was used. These tumor-types do not exhibit frequent alteration of RAS or GLI. After pooling mRNAs for each tumor type, oligo dT-primed cDNA libraries were constructed in bacteriophage lambda (Table 1). The libraries were high-titer (assessed prior to amplification on agar plates) with a mean insert size of 1.6-1.7 kb. The amplified breast cDNA library was further assessed by plaque screening for the transcription factor hBRF using a probe derived from the 5′ end of the protein coding region (bases 315-655, accession U75276). Each of the seven clones identified were derived from independent reverse transcripts, as determined by end sequencing, confirming that complexity of the library was maintained during amplification. The inserts ranged in size from 2.1-3.4 kb, and contained the entire 3′ UTR and much or all of the protein coding region intact. Three of the seven extended through the predicted initiator methionine codon, while four others were truncated further downstream. These results suggested that the library is relatively free of C-terminally truncated clones, and contains full-length cDNAs even for relatively long mRNAs. The overall abundance of hBRF mRNA has not been determined.

### EXAMPLE 10

Isolation of c-MYC and GKLF by Expression Cloning

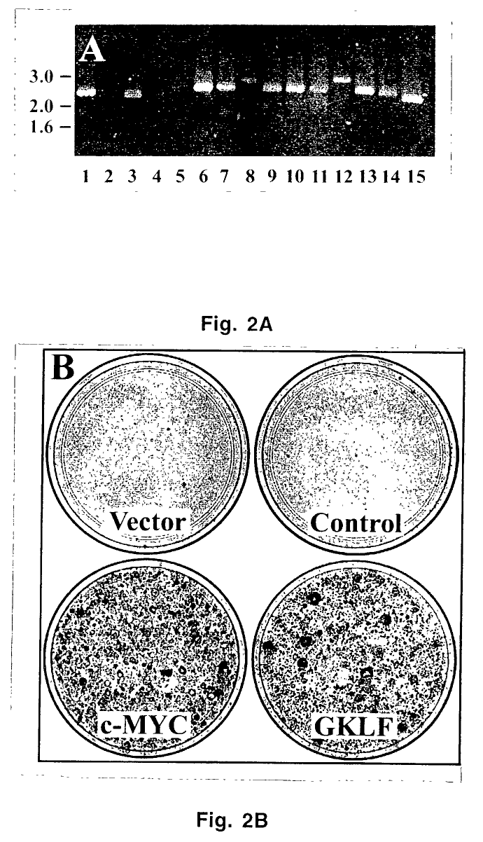

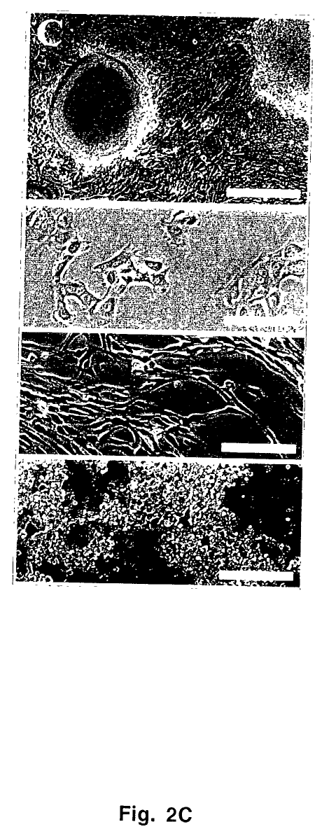

The libraries were cloned into the MMLV retroviral expression plasmid pCTV1B (27), packaged in BOSC23 cells (29), and high-titer virus supernatants were applied to RK3E cells. Fourteen foci, identified at 10-20 days post-transduction, were individually expanded into cell lines. Thirteen of these contained a single stably integrated cDNA, as indicated by PCR (FIG. 2A). Eleven of these were identified as human c-MYC by end-sequencing and restriction enzyme analysis. The C-MYC cDNA in lane 15 included the coding region and 193 bases of 5′ UTR sequence (Accession V00568). As determined by sequencing or restriction mapping, the other c-MYC cDNAs extended further 5′ (lanes 1,3,5-7,9-11,13-14), such that all of the clones contained the entire protein-coding region.

In addition, two cell lines (FIG. 2A, lanes 8 and 12) contained cDNAs coding for GKLF. Mouse and human GKLF cDNAs were previously isolated by hybridization with zinc finger consensus probes (30-32), but were not implicated as oncogenes or found to be induced during neoplastic progression. After cloning into plasmid, the sequences of these two cDNAs, termed SQC7 and SQC11, were obtained in total. As determined by comparison with multiple expressed sequence tags (ESTs) and two full-length coding sequence files in the database (Accessions U70663, AFO22184), each contained the predicted GKLF protein coding region bounded by 5′ and 3′ UTRs. An ATG in good context for translation initiation was located at base 330, with the predicted terminator codon at base 1740. Both isolates were artificially truncated at the Xho I site in the 5′ UTR during library preparation. As the transcripts had been processed using distinct AAUAAA (SEQ ID No. 7) polyadenylation signals, the cDNAs were slightly different in length and derived from independent mRNA molecules (FIG. 2A).

Sequencing revealed these two GKLF isolates to be identical within the residual 5′ UTR and throughout the coding region. A single base-pair difference in the 3′ UTR represents a PCR-induced error or a rare variant, as determined by comparison with ESTs. Comparison to a placenta-derived sequence (Accession U70663) revealed three single base-pair differences in the coding region. These differences were resolved by alignment with other sequences in the database (Accessions AF022184, AA382289) from normal tissues, indicating that the GKLF molecules obtained by expression cloning are predicted to encode the wild-type protein.

### EXAMPLE 11

Reconstitution of Transforming Activity for c-MYC and GKLF

To demonstrate transforming activity, three independent PCR products each for the c-MYC and GKLF cDNAs were cloned into the retroviral expression vector pCTV3K (27), packaged into virions, and tested for transformation of RK3E cells in vitro (FIGS. 2B and 2C, Table 2). One of the c-MYC clones (pCTV3K-SQC1) possessed greatly reduced transforming activity in multiple experiments despite similar viral titers, as determined by induction of hygromycin resistance, suggesting that an error may have been introduced during PCR. Each of the other virus supernatants carrying GKLF and c-MYC transgenes induced >1000 foci per dish compared to no foci for virus controls.

TABLE 2
Retroviral transduction of reconstituted
GKLF and c-MYC expression vectors

| | | Colony |
|---|---|---|
| | Focus assay | morphology assay |
| Plasmid | (#foci/10 cm dish) | (# transformed/total)^{d} |
| pCTV3K (vector) | 0, 0 | 0/184 |
| pCTV3K-SQC1^{a} (c-MYC) | 0, 0 | 0/232 |
| pCTV3K-SQC5 (c-MYC) | >1000, >1000 | ND |
| pCTV3K-BR1 (c-MYC) | >1000, >1000 | 81/91 (89%) |
| pCTV3K-SQC7 (GKLF) | >1000, >1000 | 91/206 (44%) |
| pCTV3K-SQC11-2^{b} | >1000, >1000 | ND |
| (GKLI) | | |
| pCTV3K-SQC11-3 (GKLF) | >1000, >1000 | ND |

^{a}pCTV3K-SQC1 is a c-MYC allele obtained by PCR that exhibited greatly reduced transforming activity compared with other alleles.
^{b}SQC11-2 and -3 are independent plasmid clones derived from the same PCR reaction (FIG. 2A, lane 12).
^{C}RK3E cells transduced with 4 mls of virus supernatant after calcium phosphate-mediated plasmid transfection of virus packaging cells.
^{d}RK3E cells transduced with 0.4 mls of thawed viral supernatant. Cells split 1:4 into selective media 30 hours later. At 2 weeks, drug-resistant colonies were fixed, stained, and examined visually for morphological transformation. Numbers indicate colonies per 10 cm dish. A duplicate transduction experiment yielded similar results. No colonies formed in control dishes that were not exposed to virus
ND, not determined.

To determine the efficiency of transformation by GKLF and c-MYC, a colony morphology assay was used as described (27). Virally transduced cells were selected in hygromycin at low confluence, and stable colonies were fixed, stained, and scored for morphological transformation by visual inspection as above for foci (Table 2). The c-MYC-transduced cells exhibited loss of contact inhibition and dense growth in 89% of colonies. The GKLF-transduced cells exhibited a transformed morphology in 44% of colonies. In comparison, a previous study showed that 70% and 40% of NIH3T3 colonies transduced by viruses carrying RAS and RAF exhibited a transformed morphology (27). Virus supernatants were likewise tested for transformation of NIH3T3 cells. Neither C-MYC nor GKLF induced morphological transformation of NIH3T3 colonies, as previously described for GLI and others (17). These results identify the RK3E assay as not only highly specific, but also sensitive to the activity of a select group of oncogenes.

In lieu of sequencing the c-MYC alleles, that wild-type c-MYC can transform RK3E cells was confirmed. A human wild-type expression vector (pSRαMSV c-MYC tk-neo) induced foci using direct plasmid transfection of RK3E cells in multiple experiments. Foci were observed at a similar frequency using known wild-type or new c-MYC isolates when analyzed in parallel. In addition, retrovirus encoding the estrogen receptor-c-MYC (wild-type) fusion protein induced morphological transformation of RK3E cells in the presence or absence of 4-hydroxy-tamoxifen (33). No effect was observed for controls (empty vector or a control containing a deletion in c-MYC residues 106-143).

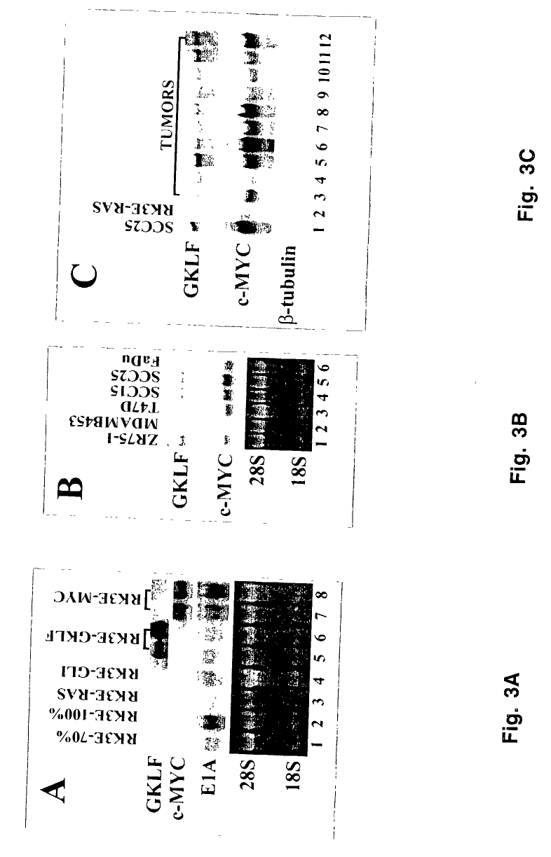

Northern blot analysis of transformed RK3E cell lines demonstrated expression of the c-MYC and GKLF vector-derived transcripts (FIG. 3A). No endogenous transcripts were detected at the stringency used in this experiment. Compared with RK3E cells at subconfluence (lane 1) or confluence (lane 2), no consistent increase of E1A transcripts was detected in cells transformed by RAS, GLI, c-MYC, or GKLF, suggesting that these genes act upon cellular targets to induce transformation.

To detect the endogenous rat GKLF transcript, reduced-stringency wash conditions and a SmaI fragment from the coding region exclusive of the C-terminal zinc fingers and with no sequence similarity to other genes in the database were used. By this approach, the apparent GKLF transcript was identified and migrated at 3.1 kb, similar to the human 3.0 kb transcript, in RK3E and all derivative transformed cell lines. A single transcript with the same mobility was detected by hybridization of the filter to full-length coding region probe. These studies revealed similar GKLF expression in RK3E and in derivatives transformed by RAS, GLI, or c-MYC. The results show that GKLF mRNA expression is not significantly altered by these other oncogenes, and is consistent with function of GKLF in an independent pathway.

Cell lines derived from foci induced by c-MYC or GKLF were further tested for tumorigenicity in athymic mice by subcutaneous inoculation at four sites for each line (Table 3) (17). Tumors were >1 cm in diameter and were scored at 2-4 weeks post-inoculation. Cells transformed by c-MYC induced tumors in 75% or 100% of sites injected (two lines tested). Three lines transformed by GKLF each induced tumors in 50-75% of sites injected. No tumors resulted from injection of RK3E cells, while a GLI-transformed cell line induced tumors in each of the four sites injected. In all, GKLF cell lines induced tumors in 8/12 injection sites, compared with 7/8 for c-MYC and 4/4 for GLI. GKLF-induced tumors also grew more slowly in vivo, reaching 1 cm in diameter by 3.4 weeks, on average, compared with 2.6 weeks for c-MYC and 3 weeks for GLI. The moderately increased latency and decreased efficiency of tumor formation for GKLF cell lines may be attributable to the intrinsic rate of proliferation for these cells (Table 3). While C-MYC, GLI, and GKLF cell lines all exhibited prolonged doubling times in vitro compared with RK3E cells, GKLF cells divided more slowly than the other transformed cell lines.

TABLE 3
Tumorigenicity of RK3E-derived cell lines in athymic mice

| | #Tumors/#Site | Tumor Latency | Doubling Time |
|---|---|---|---|
| Cell Line | Injected | in vivo (weeks)^{c} | vitro (hrs) |
| RK3E | 0/4 | — | 12.7 |
| RK3E-c-MYC BR1 | 3/4 | 3, 3, 4 | 19.1 |
| RK3E-c-MYC B^{b} | 4/4 | 2, 2, 2, 2 | 19.8 |
| RK3E-GKLF E | 3/4 | 3, 3, 3 | 33.7 |
| RK3E-GKLF F | 2/4 | 4, 4 | 27.0 |
| RK3E-GKLF G | 3/4 | 3, 3, 4 | ND |
| RK3E-GLI | 4/4 | 3, 3, 3, 3 | 18.0 |

^{a}Cell line derived from a focus identified in the original screen using a breast cancer cDNA library.
^{b}Cell line derived by transformation with the reconstituted plasmid pCTV3K-BR1.
^{c}The time required for tumors to reach 1 cm. in diameter is indicated.
ND - not determined

### EXAMPLE 12

Northern Blot Analysis of Tumors and Tumor-Derived Cell Lines

Human tumors and cell lines by Northern blot analysis of total RNA (FIGS. 3B and 3C) was examined. GKLF expression in breast or squamous cell carcinoma cell lines was variable, with increased expression in the breast tumor line ZR75-1 and the squamous cell lines SCC15 and SCC25 (FIG. 3B). In human squamous cell carcinomas microdissected to enrich for tumor cells, GKLF expression was detected in each of ten primary or metastatic tumors analyzed, with expression levels comparable to that for the cell line SCC25 (FIG. 3C). The results suggest that GKLF represents a potent transforming activity that is consistently expressed in tumors as well as in tumor-derived cell lines. As GKLF was isolated from cell lines that express the gene at a level found in tumors in vivo, the results suggest that GKLF may represent a major transforming activity in tumors as well as in cell lines.

### EXAMPLE 13

Gene Copy Number of c-MYC and GKLF

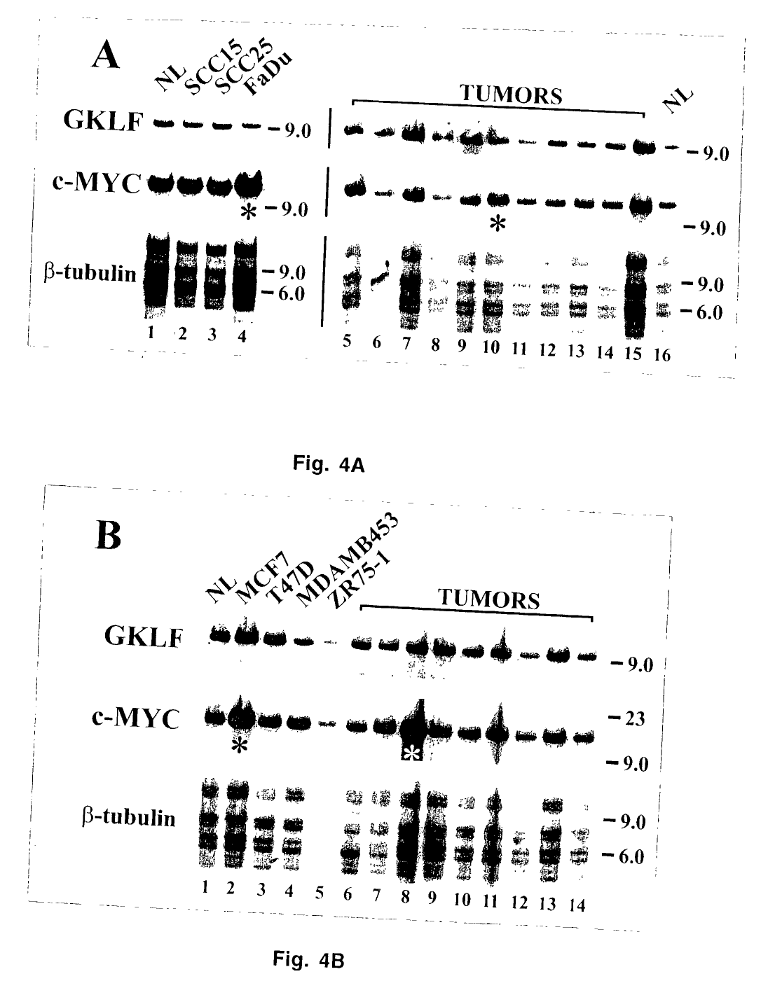

c-MYC has been shown to be activated by gene amplification in ˜10% of oral squamous cancers, and may be activated in these or other tumors by genetic alteration of WNT-APC-_-catenin pathway components (6,34-37). To determine whether expression of GKLF in cell lines and tumors is likewise associated with gene amplification, southern blot analysis (FIGS. 4A and 4B) was performed. Filters were sequentially hybridized to GKLF, c-MYC and β-tubulin. Increased copies of c-MYC were identified in two cell lines used for library construction, FaDu and MCF7. Increased hybridization to c-MYC was likewise observed for one of eleven oral squamous cell carcinomas (FIG. 4A, lane 10) and for one of nine breast carcinomas (FIG. 4B, lane 8). These results are consistent with the published frequencies of c-MYC amplification for these tumor types (34,35,38). No copy number gains of GKLF were observed, indicating that other mechanisms may contribute to expression of GKLF in tumors. The same may be true for c-MYC, as gene amplification in FaDu cells was associated with reduced expression compared with other oral cancer cell lines (FIG. 3B).

### EXAMPLE 14

GKLF Expression is Activated Early During Tumor Progression in Vivo

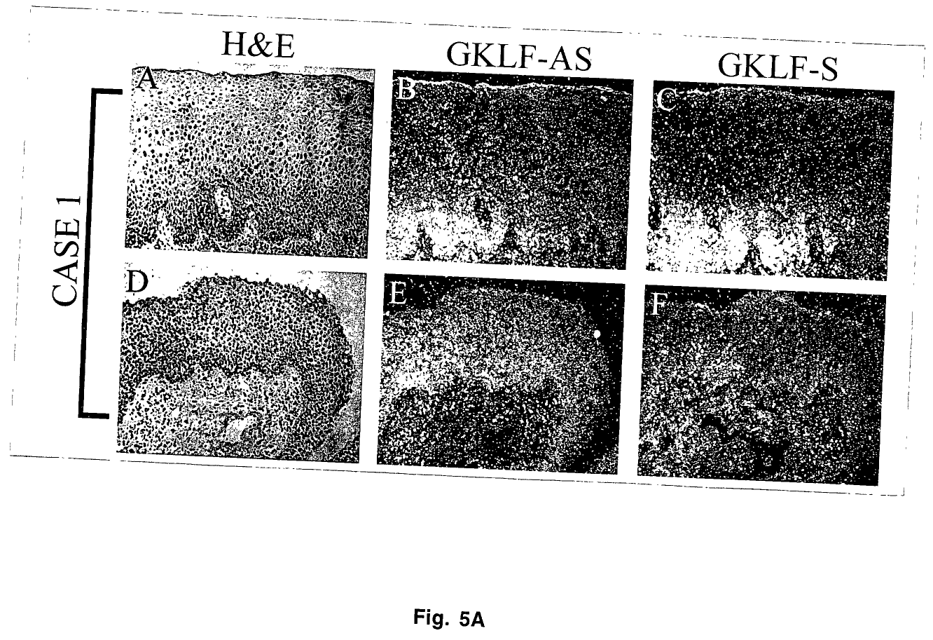

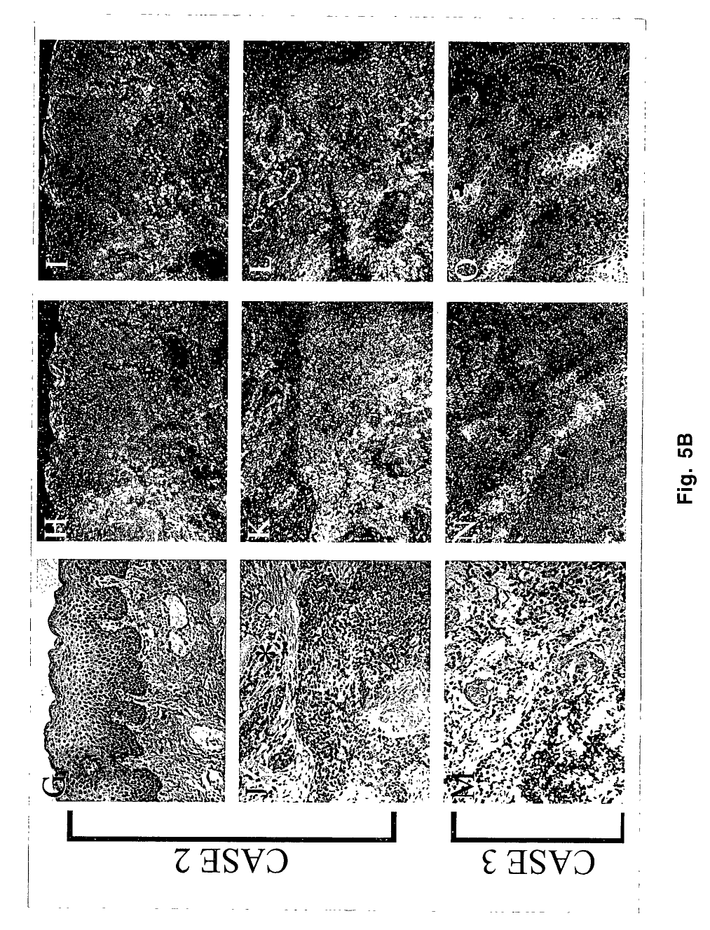

Previously, expression of c-MYC was found to be up-regulated consistently in dysplastic oral mucosa and in squamous cell carcinomas, and tumors with the highest levels of c-MYC expression were associated with the poorest clinical outcome (36,39-41). To determine how GKLF mRNA expression is altered during tumor progression, squamous cell carcinoma of the larynx and adjacent uninvolved epithelium from the same tissue blocks were analyzed using ³⁵S-labelled riboprobes by in situ hybridization analysis. In apparently normal epithelium, GKLF expression was detected in the spinous layer above the basal and parabasal cells (9 specimens analyzed) (FIGS. 5A-C, 5G-I; Table 4). No specific GKLF expression was detected in the basal or parabasal cells or in the underlying dermis. In contrast, a sense control probe produced grains at a much-reduced frequency in a uniform fashion across the epithelium. GAPDH expression served as a positive control, and was detected diffusely throughout the entire epithelium. The pattern of GKLF expression is identical to the pattern in normal mouse skin (32).

TABLE 4
Expression of GKLF in oral epithelium and tumors

| | Histopatholog | Tissue Source | Method | |
|---|---|---|---|---|
| Cas | (U, D, P, M)^{b} | (PE/FF)^{c} | (N/ISH)^{d} | GKLF express |
| 1 | U, D, P | PE | ISH | D, P > U |
| 2 | U, D | PE | ISH | D > U |
| 2 | U, P | PE | ISH | P > U |
| 3 | M | FF | ISH | + |
| 4 | U, D | PE | ISH | D > U |
| 5 | P | FF | N, ISH | + |
| 6 | M | FF | N, ISH | + |
| 7 | P | FF | ISH | + |
| 8 | P | FF | N, ISH | + |
| 9 | D, P | PE | ISH | D, P+ |
| 10 | M | PE | ISH | + |
| 11 | U, D, P | PE | ISH | D, P > U |
| 12 | U, D | PE | ISH | D > U |
| 12 | U, D, P | PE | ISH | D, P > U |
| 13 | U | PE | ISH | + |
| 13 | P | PE | ISH | + |
| 14 | P | PE | ISH | + |
| 14 | M | PE | ISH | + |
| 15 | D | PE | ISH | + |
| 15 | D | PE | ISH | + |
| 15 | D, P | PE | ISH | D, P+ |
| 16 | U, D, P | PE | ISH | D, P > U |
| 16 | M | PE | ISH | + |
| 17 | D, P | PE | ISH | D, P+ |
| 18 | P | FF | N | + |
| 19 | P | FF | N | + |
| 20 | M | FF | N | + |
| 21 | P | FF | N | + |
| 22 | M | FF | N | + |
| 23 | M | FF | N | + |
| 24 | P | FF | N | + |

^{a}Each row corresponds to a tissue specimen. Levels of gene expression indicate changes identified within, rather than between, single tissue sections. For some cases multiple specimens isolated during the same surgical procedure were analyzed. ISH results were confirmed by analysis of sections in duplicate.
^{b}U, uninvolved or normal-appearing epithelium;
D, dysplastic epithelium;
P, primary tumor;
M, metastatic tumor.
^{c}PE, paraffin-embedded;
FF, fresh-frozen.
^{d}N, Northern;
ISH, mRNA in situ hybridization.
^{e}D, P > U indicates increased expression in dysplasia and primary tumor compared with uninvolved epithelium in the same section.
D, P+ indicates expression in both dysplasia and adjacent primary tumor.

For each of 12 specimens analyzed, dysplastic epithelium exhibited increased GKLF expression throughout the epithelium (FIGS. 5D-F; Table 4, cases 1, 2, 4, 9, 11, 12, 15-17). In contrast to results obtained in normal-appearing epithelium, there was no reduction of expression in the basal and parabasal layers compared with superficial layers. For tissue sections that contained both uninvolved epithelium and adjacent dysplastic epithelium, the overall level of GKLF expression in dysplastic epithelium was prominently elevated compared with the GKLF-positive cell layers in uninvolved epithelium (FIGS. 5B, 5E, and 5H; Table 4, cases 1, 2, 4, 11, 12, and 16). These results suggest that GKLF expression is qualitatively and quantitatively altered in dysplasia, that exclusion of GKLF from the basal and parabasal cell layers is lost early during neoplastic progression, and that GKLF exhibits properties of an oncogene not only in vitro but also in vivo.

As shown by northern blot analysis, GKLF transcripts are consistently present in tumor-derived mRNA (FIG. 3C, Table 4). To determine whether GKLF is expressed in tumor cells, laryngeal squamous cell carcinomas was examined by mRNA in situ hybridization. Expression was detected in each primary (13 cases) or metastatic (5 cases) tumor examined (FIGS. 5J-O; Table 4), with all or nearly all tumor cells associated with silver grains. The level of expression was somewhat heterogeneous, with higher levels found in the periphery and in nodules of tumor containing centrally necrotic cells or keratin pearls. As for dysplastic epithelium, expression in tumor cells was consistently elevated compared with uninvolved epithelium in the same sections (FIGS. 5H and 5K; Table 4, cases 1, 2, 11, 12, 16). However, expression in tumor cells was not higher than in dysplastic epithelium (cases 1, 9, 11, 12, 15-17). For several cases expression in the most dysplastic epithelium was higher than in adjacent GKLF-positive tumor, suggesting that GKLF expression is specifically activated during the transition from normal epithelium to dysplasia, prior to invasion or metastasis.

### EXAMPLE 15

Identification of Transforming Oncogenes in Oral Cancer

A cDNA expression library was prepared using mRNA from human oral cancer cell lines. Using retroviral transduction, 4 million independent cDNAs were stably expressed in RK3E cells. 14 foci were identified. Single human cDNAs were identified in each of the clones using long PCR. 12 of these were c-MYC alleles truncated in the 5′ untranslated region. Two were independent, full-length, wild type alleles of a novel oncogene, SCC7, encoding a poorly characterized putative transcription factor not previously implicated in transformation. Expression vectors reconstituted using c-MYC or SCC7 PCR products induced hundreds of foci per dish. By Northern analysis, high level expression of SCC7 was observed in oral and breast cancel cell lines (5/6 tested). Expression of the endogenous rat SCC7 mRNA was upregulated in transformed rat kidney cells compared with immortalized parental cells. Cells transformed by c-MYC and SCC7 exhibited expression of the respective vector-derived mRNA and were tumorigenic in athymic mice. Expression of E1a was not altered by any of the oncogenes. The results demonstrate that known and novel oncogenes can be rapidly identified in a specific fashion using epithelial-like host cells, and show that SCC7, c-MYC, RAS, and GLI can each transform cells in cooperation with adenovirus E1a in vitro. By analogy with c-MYC, RAS and GLI, activation of SCC7 may likewise contribute to tumor progression in vivo.

### EXAMPLE 16

mRNA Expression

In situ hybridization was conducted, using sense and antisense [³⁵S]-labeled riboprobes prepared by in vitro transcription of a cDNA fragment corresponding to the 3′ untranslated region of human GKLF. A GAPDH antisense probe corresponding to bases 366-680 (Accession M33 197) was synthesized using a commercially available template (Ambion, Inc., Austin, Tex.). High stringency washes were in 0.1×SSC and 0.1% (v/v) 2-mercaptoethanol at 58° C. for GKLF or 68° C. for GAPDH. Slides were coated with emulsion and exposed for 14 days. Results were scored using a 0.0 to 4.0 scoring system, where 0.0 indicated only nonspecific background and 1.0 corresponded to an average of four grains per nucleus.

Breast adenocarcinoma cell lines were obtained from the American Type Culture Collection (Manassus, Md.). Human mammary epithelial cells were described previously and were cultured in mammary epithelial basal media (Clonetics Corp., Walkersville, Md.) (61). Extracts were prepared from exponentially growing cells at 70% confluence, and total RNA isolation and Northern blot analysis were performed.

### EXAMPLE 17

Isolation of an Anti-GKLF Monoclonal Antibody

The region of the human GKLF cDNA encoding residues 479-1197 (accession AF105036) was cloned into plasmid pET-32a-ZFP4 and expressed i n E. coli BL21(DE3) bacteria as a His-tagged protein. Protein was purified from the bacteria after induction with IPTG using a His-Trap Ni-agarose column (Amersham Pharmacia Biotech, Piscataway, N.J.) and eluted with 500 mM imidazole. Purified protein was used to immunize two mice, and lymphocytes were fused with murine myeloma cells (PX63-Ag8.653) as described previously (62). Hybridomas that were immunoreactive in an ELISA assay for the purified antigen were cloned and recloned by limiting dilution. Positive clones were identified by ELISA, and an IgG₁ antibody (αGKLF) was purified from ascites on a protein A affinity column.

### EXAMPLE 18

Immunohistochemistry

Tissues were fixed in neutral buffered formalin and embedded in paraffin. Deparaffinized tissue sections were incubated with αGKLF at a concentration of 1.0 μg/ml for 1 hr at room temperature, and processed as described (63). Immunodetection was performed using a biotinylated secondary antibody, streptavidin-horseradish peroxidase detection system (Signet Laboratories, Dedham, Mass.), and the chromogenic substrate diaminobenzidine (Biogenex, San Ramon, Calif.). Sections were counterstained with hematoxylin. Results were scored by using a 0.0 to 4.0 scoring system, wherein 4.0 corresponds to a saturated signal (64).

### EXAMPLE 19

Statistical Analyses

Paired t-tests were utilized to compare the differences in expression in breast epithelial cells at various stages of tumor progression (65). Pearson correlation coefficients were used to compare results obtained by in situ hybridization to those obtained for the same cases using immunohistochemistry.

### EXAMPLE 20

GKLF mRNA Expression is Upregulated During Breast Tumor Progression

Previously, SAGE analysis of purified normal breast epithelial cells detected GKLF transcripts at an abundance of 40 tags per million (66, 67). In the present study, Northern blot analysis of breast tumor cell lines revealed the presence of GKLF transcripts. Using sense and antisense [³⁵S]-labeled riboprobes, the expression of GKLF mRNA was examined in 31 cases of carcinoma of the breast. Specificity of hybridization was determined by using the sense probe as a negative control or by hybridization of the antisense probe to human foreskin, in which GKLF was specifically detected in suprabasal epithelial cells (not shown).

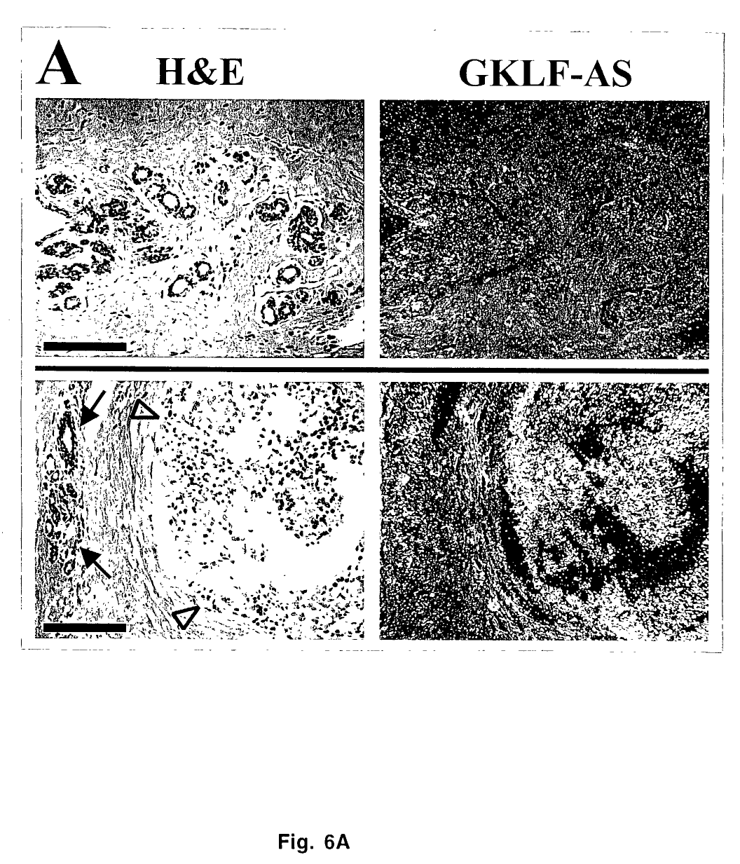

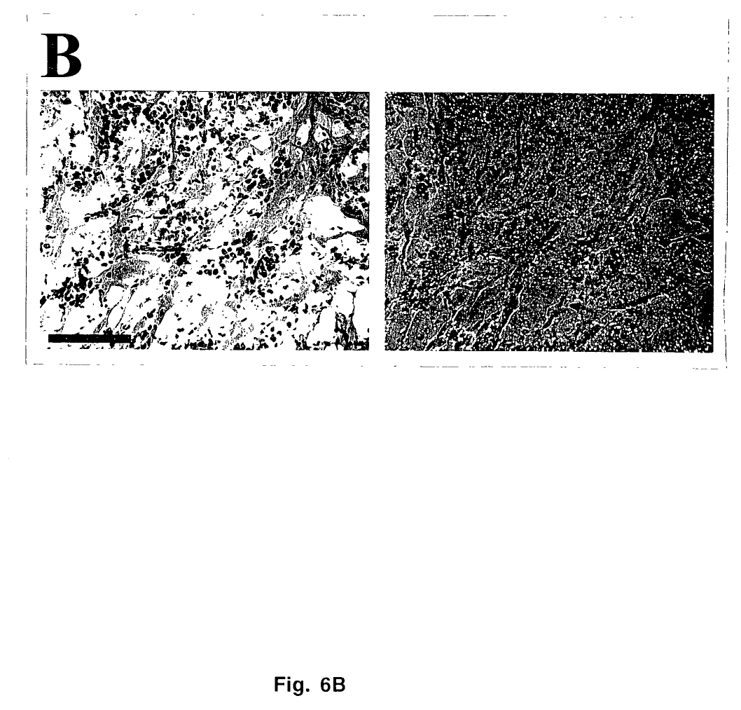

Expression of GKLF was detected in malignant cells in 21 of 31 cases of ductal adenocarcinoma (68%, FIG. 6, Table 5). For several cases that exhibited no detectable expression of GKLF, prominent expression of the housekeeping gene GAPDH was observed, indicating that overall mRNA integrity was maintained and that failure to identify GKLF transcripts may reflect reduced levels of expression. GKLF expression was increased in malignant cells of 14 of 19 cases that contained adjacent uninvolved epithelium (FIG. 6A). For 7 of these 14 cases, no specific signal was detected in adjacent uninvolved epithelium. In the other 7 cases, expression was detected in both uninvolved and malignant cells, with expression of GKLF in malignant cells increased by 3-5 fold compared with uninvolved epithelium. Within tumors, expression of GKLF was specific to malignant cells, with little or no expression detected in stromal components (FIG. 6B).

TABLE 5
mRNA in situ hybridization analysis of GKLF in tumors^{a}

| | | Carcinoma of the Breast | | | | |
|---|---|---|---|---|---|---|
| | | GKLF-AS | | | | |
| CASE | PE/FF | U | D | T | GKLF-S | GAPDH-AS |
| 1 | FF | 0. | 2.5 | − | 0.0 | + |
| 2 | FF | − | − | 2. | 0.0 | + |
| 3 | FF | 0. | − | 1. | 0.0 | + |
| 4 | FF | − | − | 0. | 0.0 | + |
| 5 | FF | − | − | 0. | 0.0 | NT |
| 6 | FF | − | − | 0. | 0.0 | NT |
| 7 | FF | − | 2.0 | 2. | 0.0 | NT |
| 8 | FF | 0. | 1.0 | 1. | 0.0 | NT |
| 9 | FF | − | − | 0. | 0.0 | NT |
| 10 | FF | − | − | 0. | 0.0 | NT |
| 11 | FF | − | − | 0. | 0.0 | NT |
| 12 | FF | − | − | 0. | 0.0 | NT |
| 13 | FF | 0. | − | 0. | 0.0 | NT |
| 14 | FF | − | − | 0. | 0.0 | NT |
| 15 | PE | − | − | 1. | NT | + |
| 16 | PE | 0. | − | 1. | NT | + |
| 17 | PE | 0. | − | 1. | NT | + |
| 18 | PE | 0. | − | 2. | NT | + |
| 19 | PE | − | − | 0. | NT | + |
| 20 | PE | 1. | 2.0 | 1. | NT | + |
| 21 | PE | 0. | − | 1. | NT | + |
| 22 | PE | 0. | 2.0 | 2. | NT | + |
| 23 | PE | 1. | − | 1. | 0.0 | + |
| 24 | PE | 0. | 1.0 | 1. | 0.0 | + |
| 25 | PE | 0. | 1.2 | 1. | 0.0 | + |
| 26 | PE | 0. | 1.5 | 1. | 0.0 | + |
| 27 | PE | 0. | 0.0 | 0. | 0.0 | + |
| 28 | PE | 0. | 0.0 | 0. | 0.0 | + |
| 29 | PE | 0. | 0.0 | 0. | 0.0 | + |
| 30 | PE | 0. | 1.0 | 1. | 0.0 | + |
| 31 | PE | 0. | 1.0 | 1. | 0.0 | 0.0 |

| | | Carcinoma of the Prostate | | | | |
|---|---|---|---|---|---|---|
| | | GKLF-AS | | | | |
| CASE | PE/FF | U | PIN | T | GKLF-S | GAPDH-A |
| 1 | PE | 1. | − | 0. | NT | + |
| 2 | PE | − | − | 0. | NT | + |
| 3 | PE | 1. | − | 1. | NT | + |
| 4 | PE | 1. | 1.0 | 0. | NT | 0.0 |

^{a}Results obtained for sense (S) or antisense (AS) probes are presented. Scoring of GKLF used a scale of 0.0 to 4.0, whereas GAPDH was scored as detected (+) or undetected (0.0). Numbers indicate the level of gene expression for histologically distinct tissue within the same section.
A dash (−) indicates that no tissue in the section exhibited the specific histopathologic feature.
PE, paraffin-embedded;
FF, fresh-frozen;
U, uninvolved or morphologically normal epithelium; D, ductal carcinoma in situ;
PIN, prostatic intraepithelial neoplasia;
T, invasive tumor cells;
NT, not tested.

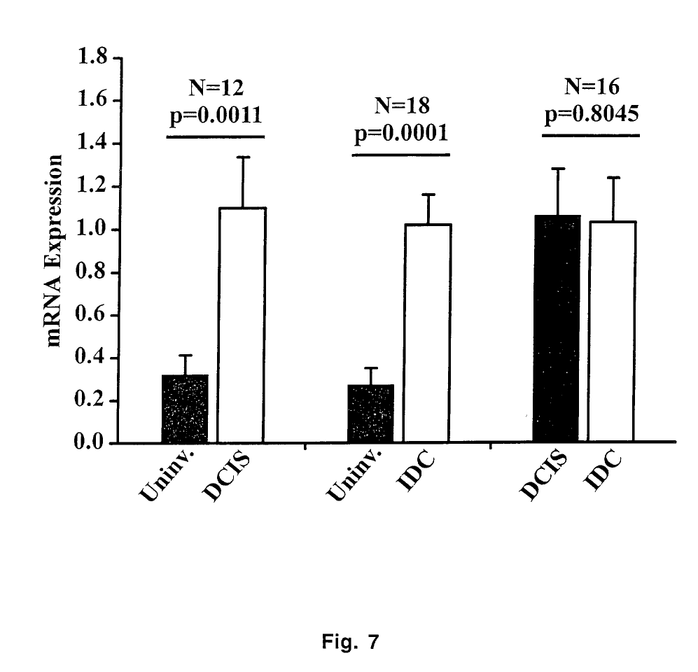

GKLF expression in DCIS was not significantly different from invasive carcinoma, but expression in both lesions was higher than for uninvolved breast epithelium (Table 5, FIG. 7). In contrast to results obtained in breast tumors, examination of several cases of prostatic carcinoma revealed equal or reduced expression in tumor cells compared with adjacent uninvolved glandular epithelial cells (Table 5). In summary, the results suggest that GKLF mRNA expression is activated in approximately two-thirds of breast carcinomas, and that expression in positive cases is consistently induced in DCIS prior to invasion.

### EXAMPLE 21

Characterization of a GKLF-Specific Monoclonal Antibody

An IgG₁ isotype antibody raised against bacterially-expressed GKLF was subsequently referred to as αGKLF. Immunoblot analysis of GKLF-transformed RK3E cells and control cell lines detected a single protein species of 55 kDa, consistent with the predicted size of the full-length polypeptide (data not shown). Compared with RK3E cells or control cell lines transformed by other oncogenes, apparent GKLF abundance was increased by several-fold in each of two cell lines transformed by the human expression vector. The epitope recognized by the antibody may be denaturation sensitive, as a signal was obtained only after overnight exposure of autoradiographic film using a standard chemiluminescence protocol. The antibody was not sufficiently sensitive to detect GKLF by immunoblot analysis of extracts of human tumor cell lines that express the endogenous GKLF mRNA.

The cell type- and tumor type-specific patterns of GKLF mRNA expression were utilized to examine the specificity of αGKLF in immunohistochemical assays. These patterns can be summarized as follows. Human GKLF mRNA is detected by in situ hybridization in differentiating cells of oral epithelium, and is markedly elevated in oral tumors. The mRNA is not detected in morphologically normal basal or parabasal cells, particularly within epidermal pegs that extend further into the submucosa. Mouse GKLF mRNA is similarly found to be more highly expressed in superficial, differentiating cells of the skin and gut, and is reduced or absent in basal epithelial cells in both tissues (30,32,68). In contrast to human oral and breast cancer, GKLF mRNA expression is reduced in mouse colorectal tumors compared with normal epithelium (51), and is similarly reduced in human colorectal cancer as indicated by SAGE (66).

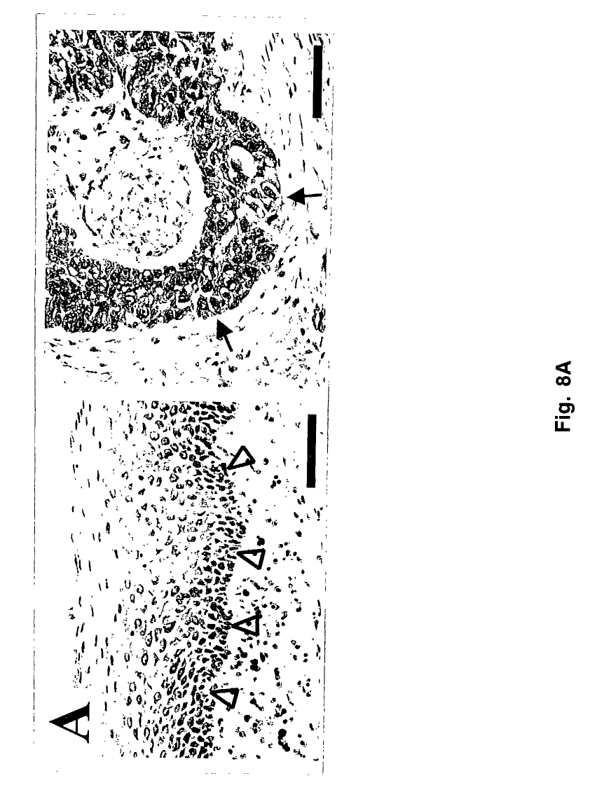

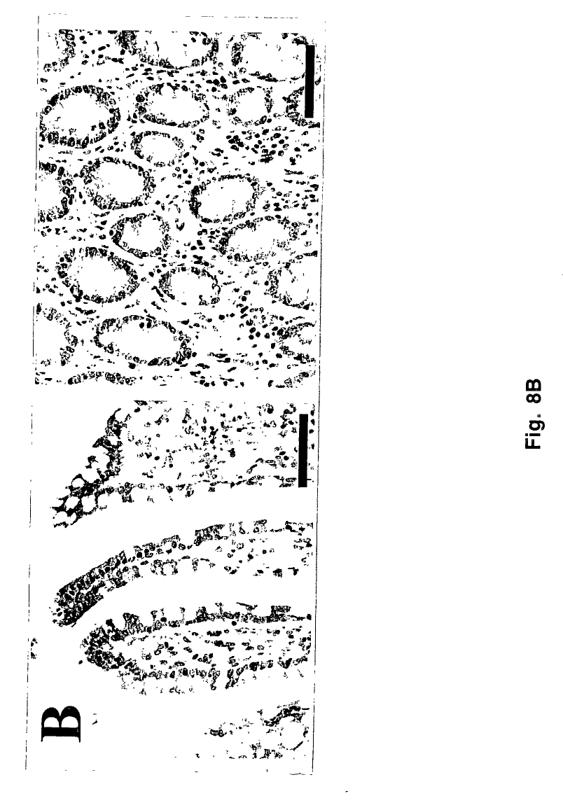

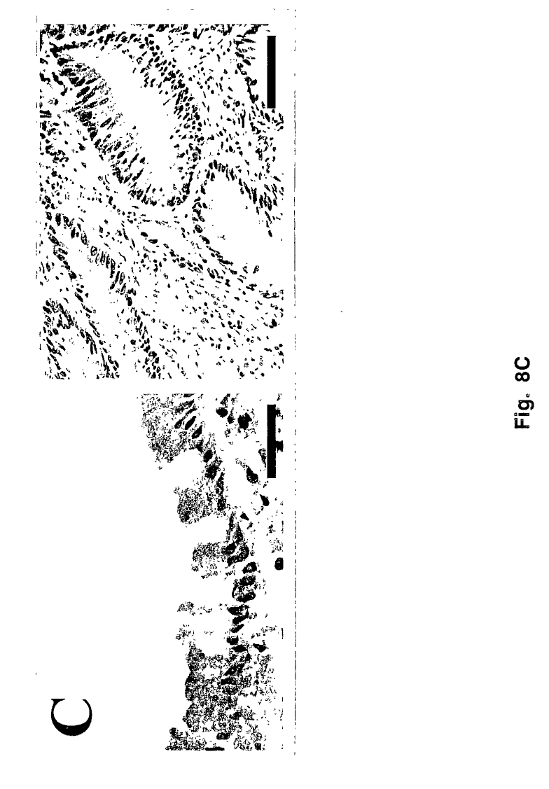

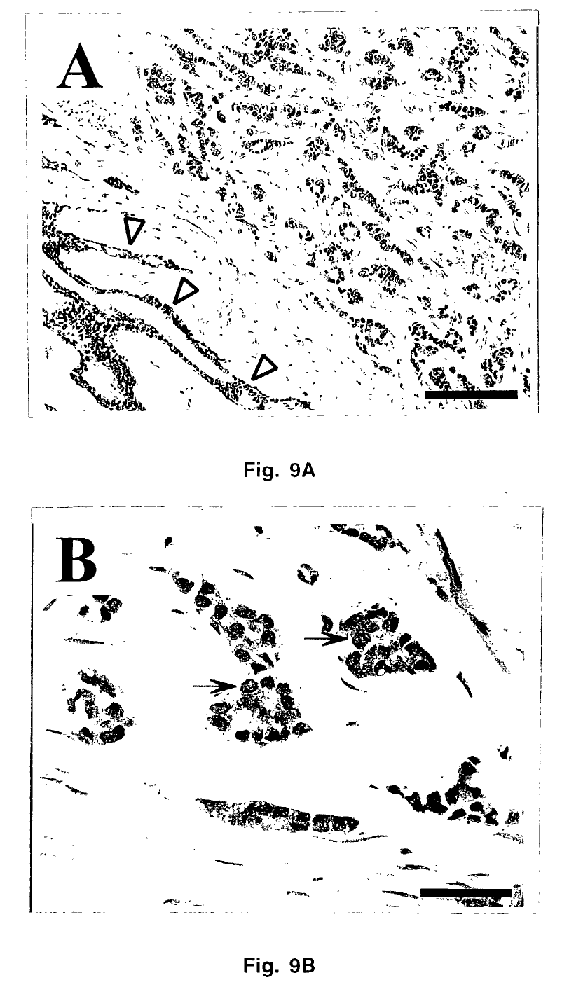

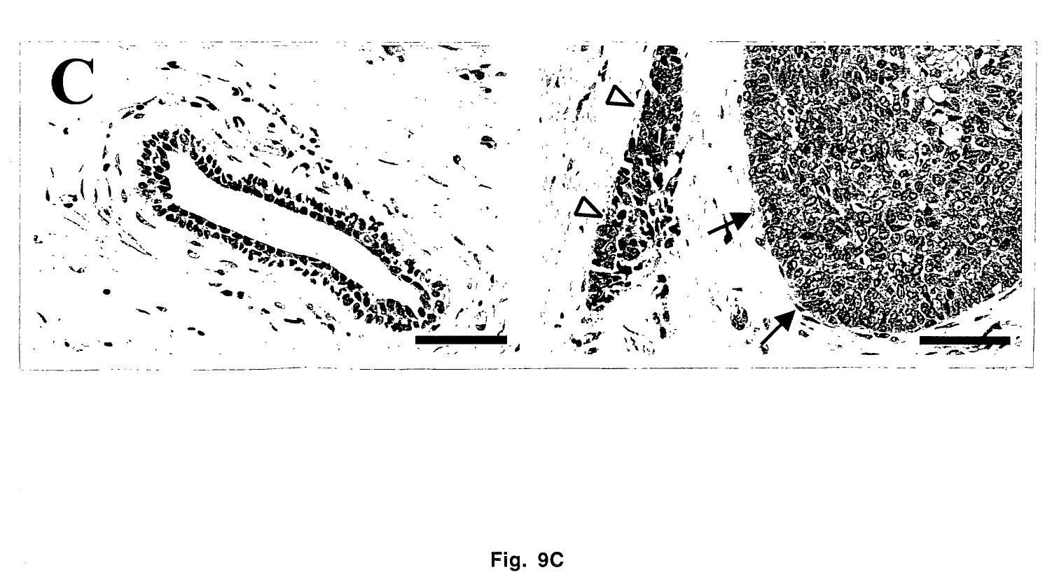

The staining pattern of αGKLF exhibited a strict concordance with detection of GKLF mRNA (FIGS. 8-9, Table 6). In positive tissues, αGKLF exhibited a mixed nuclear and cytoplasmic staining pattern. For uninvolved epithelium, DCIS, and invasive carcinoma alike, the average cytoplasmic staining was 1.8-2.5 fold greater than nuclear staining, suggesting that subcellular localization was not altered during tumor progression in any consistent fashion. Cytoplasmic staining was subsequently used as at more sensitive indicator of overall expression.

In several samples of skin or oral squamous epithelium, αGKLF bound specifically to differentiating suprabasal epithelial cells (FIG. 8A). Compared with adjacent uninvolved epithelium, staining was markedly increased in malignant cells for each of several cases of squamous cell carcinoma, with little or no staining of stromal components of the tumor. Likewise, staining was increased in superficial cells compared to cells deeper within epithelial crypts of the small bowel (FIG. 8B) or large bowel (Table 6, P=0.043). In contrast to oral and breast tumors, staining was reduced in tumor cells compared with adjacent superficial epithelial cells for each of four cases of human colorectal adenoma or carcinoma examined (FIG. 8C, Table 6, P=0.027).

TABLE 6
Immunohistochemical analysis of GKLF in tumors^{a}.

| | | Carcinoma of the Breast | | | | | |
|---|---|---|---|---|---|---|---|
| | | Uninvolved | | DCIS | | Invasive tumor ce | |
| | | | Cyto- | | Cyto- | | Cyto- |
| CASE | PE/FF | Nucleus | plasm | Nucleus | plasm | Nucleus | plasm |
| 23 | PE | 0.25 | 0.45 | — | — | 0.35 | 0.55 |
| 24 | PE | 0.50 | 1.30 | 1.00 | 1.30 | 1.00 | 1.30 |
| 25 | PE | 0.65 | 0.95 | 0.45 | 1.40 | 0.38 | 1.35 |
| 26 | PE | 0.18 | 0.75 | 0.03 | 1.20 | 0.12 | 1.05 |
| 27 | PE | 0.10 | 1.30 | 0.00 | 1.10 | 0.05 | 0.50 |
| 28 | PE | 0.10 | 0.30 | — | — | 0.35 | 0.20 |
| 29 | PE | 0.00 | 0.00 | 0.10 | 0.75 | 0.05 | 0.75 |
| 30 | PE | 0.00 | 0.20 | 0.10 | 1.05 | — | — |
| 31 | PE | 0.00 | 0.10 | 0.65 | 0.65 | 0.70 | 1.15 |
| 32 | PE | 0.25 | 0.55 | 0.55 | 0.75 | 0.42 | 0.85 |
| 33 | PE | 0.80 | 0.45 | — | — | 0.50 | 1.25 |
| 34 | PE | 0.18 | 0.50 | — | — | 0.45 | 1.15 |
| 35 | PE | 0.30 | 0.35 | 0.60 | 1.60 | 0.65 | 1.50 |
| 36 | PE | 0.00 | 0.05 | 0.55 | 1.70 | 0.75 | 1.00 |
| 37 | PE | 0.70 | 0.60 | — | — | 1.65 | 1.80 |
| 38 | PE | — | — | 0.00 | 0.90 | 0.00 | 1.50 |
| 39 | PE | 0.55 | 0.70 | 0.75 | 0.85 | 1.75 | 1.75 |
| 40 | PE | 0.35 | 0.50 | 0.75 | 0.90 | 0.75 | 0.85 |

| | | Colorectal carcinoma | | | | | |
|---|---|---|---|---|---|---|---|
| | | Normal | | | | | |
| | | Superficial^{b} | | | | Tumor^{d} | |
| | | | Cyto- | Normal Deep^{c} | | | Cyto- |
| CASE | PE/FF | Nucleus | plasm | Nucleus | Cytopla | Nucleus | plasm |
| 1 | PE | 0.45 | 1.00 | 0.25 | 0.05 | 0.00 | 0.85 |
| 2 | PE | 0.40 | 0.60 | 0.40 | 0.25 | 0.20 | 0.35 |
| 3 | PE | 0.15 | 1.15 | 0.30 | 0.80 | 0.25 | 0.85 |
| 4 | PE | 0.00 | 1.30 | 0.00 | 0.15 | 0.00 | 0.80 |
| 5 | PE | — | — | — | — | 0.00 | 0.65 |

a Immunohistochemical scores indicate the intensity of staining of histologically distinct tissue within the same section.
A dash (-) indicates that no tissue in the section exhibited the specific histopathologic feature.
PE, paraffin-embedded;
FF, fresh-frozen; DCIS, ductal carcinoma in situ.
^{b}Differentiating epithelial cells located in the superficial portion of intestinal mucosa.
^{c}Epithelial cells deep within intestinal mucosa. ^{d} Analysis included both adenomas and adenocarcinomas.

### EXAMPLE 22

Expression of GKLF Protein is Increased During Neoplastic Progression in the Breast

Eighteen cases were tested for GKLF expression by immunohistochemistry (Table 6, FIG. 9). Nuclear and cytoplasmic staining of normal breast epithelium, DCIS, and invasive carcinoma were semi-quantitatively assessed. Low-level staining of tumor cells was observed for six cases (e.g., cytoplasmic staining ranging from 0.20 to 0.85), with eleven cases exhibiting higher-level staining (e.g., cytoplasmic staining ranging from 1.00 to 1.75). These results are consistent with detection of the mRNA in approximately two-thirds of tumors by in situ hybridization. For cases 23-31, which were analyzed by both in situ hybridization and immunohistochemical staining, results of the two methods exhibited a close correlation that reached statistical significance for invasive carcinoma cells (N=8, coefficient=0.77, P=0.024). In DCIS, the correlation was moderate even though the sample number was small (N=7, coefficient=0.43). Perhaps due to the overall lower level of expression in uninvolved tissue, the correlation was weakest in uninvolved ducts. Minor differences observed for the two methods may be attributed to differences in sensitivity and specificity, to false negative results due to partial degradation of mRNA in some surgical samples, or to analysis of non-serial sections of the same tissue block.

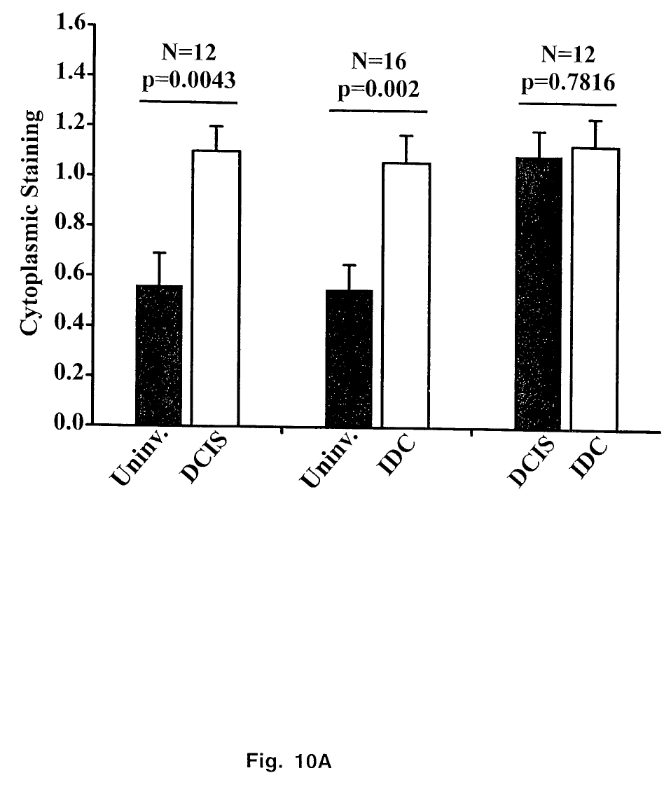

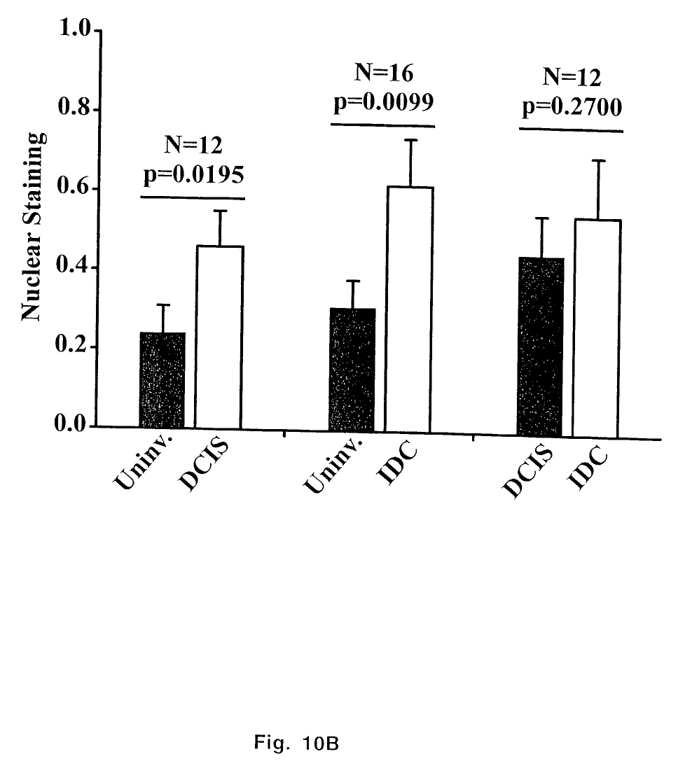

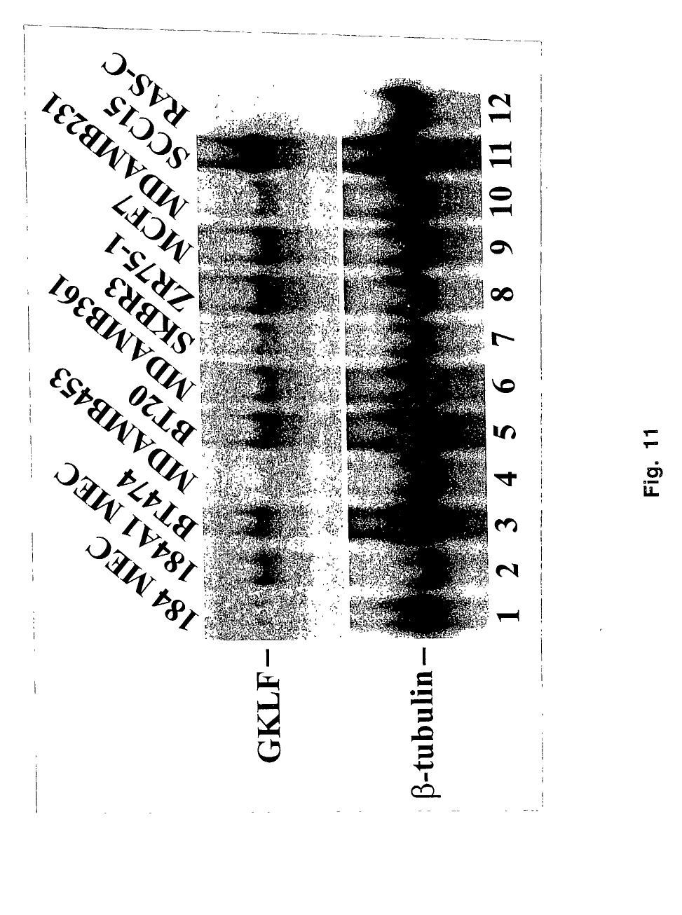

Apparent GKLF expression as determined by nuclear or cytoplasmic immunostaining was increased in both DCIS and invasive carcinoma compared with uninvolved ducts (Table 6, FIG. 10). For morphologically normal ducts, staining of myoepithelial cells was not significantly different from that of luminal epithelial cells (P=0.303, data not shown). However, staining of neoplastic cells in DCIS was significantly increased compared with myoepithelial cells within the same ducts (P=0.0001), consistent with other studies indicating similarities between tumor cells and luminal epithelial cells (69).

### EXAMPLE 23

Analysis of GKLF in Cultured Breast Epithelial Cells

Northern blot analysis of breast tumor cell lines revealed variable levels of GKLF expression relative to a tubulin control. GKLF expression was high in MCF7 and ZR75-1, intermediate in BT474, BT20, MDAMB361, and SKBR3, and reduced in MDAMB453 and MDAMB231. Thus, expression in six of eight breast tumor-derived cell lines was increased relative to 184 cells, an HMEC population of finite life-span derived from normal breast tissue following reduction mammoplasty (lane 1). Expression was similarly increased in 184A1 cells (33). These immortalized cells were derived from 184 cells by treatment with benzo(a)pyrene. They are wild-type for p53 and p105^{Rb} and are anchorage-dependent and non-tumorigenic in animals. The results obtained for breast tumor cell lines support the conclusion that GKLF expression is upregulated at the mRNA level in most breast tumors, while activation in 184A1 cells is consistent with identification of GKLF induction as an early event.

Discussion

The results demonstrate that cells with an epithelial phenotype can be used for identification of transforming activities present in carcinoma-derived cell lines. The assay repeatedly identified two genes, and none of the isolated cDNAs were artificially truncated or rearranged within the protein coding region. This indicates that transformation of these cells is unusually specific to a few pathways or genes, including c-MYC, GKLF, RAS, and GLI. c-MYC, RAS, and GLI are directly or indirectly activated by genetic alterations in diverse carcinoma types during tumor progression in vivo (9,10,42-44). For both breast and oral squamous carcinoma, the tumor-types analyzed in this study, c-MYC gene amplification is one of the more frequent oncogene genetic alterations and is observed in 10-15% of cases. By analogy, novel oncogenes identified by the RK3E assay may be directly activated in neoplasms through gain-of-function mutations or indirectly activated by loss-of-function genetic alterations.

The retroviral vectors used in this study for transduction of NIH3T3 cells were developed by Kay and colleagues (27). Using the NIH3T3 line, they isolated 19 different cDNAs encoding 14 different proteins. Known oncogenes were isolated including raf-1, lck, and ect2. Other known genes included phospholipase C-γ₂, β-catenin, and the thrombin receptor . In addition to the known genes, seven novel cDNAs were isolated, including several members of the CDC24 family of guanine nucleotide exchange factors. Only the thrombin receptor was isolated more than once, and many of the 14 different genes identified were truncated within the protein coding region. The diversity of cDNAs isolated in the NIH3T3 assay is in contrast to results obtained in the current study. The specificity of the RK3E assay may be attributable to the “tumor suppressor” activity of the E1A oncogene (28,45). Although E1A antagonizes p105^{Rb} and immortalizes primary cells, it also induces epithelial differentiation in diverse tumor types, including sarcoma, and suppresses the malignant behavior of tumor cells in vivo.

GKLF was previously isolated by hybridization to zinc finger probes (30-32). The human gene is located at chromosome 9q31 and is closely linked to the autosomal dominant syndrome of multiple self-healing squamous epitheliomata (MSSE) (31,32,46,47). Affected individuals develop recurrent invasive but well-differentiated tumors morphologically similar to squamous carcinoma that spontaneously regress. Although GKLF has been proposed as a candidate tumor suppressor gene relevant to multiple self-healing squamous epitheliomata (32), the results suggest that activating mutations could account for the syndrome.

GKLF encodes a nuclear protein that functions as a transcription factor when bound to a minimal essential binding site of 5′-^{G}/_{A}^{G}/_{A}GG^{C}/_{T}G^{C}/_{T}-3′ (SEQ ID No. 8) (48). The 470 residue polypeptide exhibits modular domains that mediate nuclear localization, DNA binding, and transcriptional activation or repression (31,32,49,50). In mice, GKLF expression is found predominately in barrier epithelia including mucosa of the mouth, pharynx, lung, esophagus, and small and large intestine (30,32). A role for GKLF in differentiation or growth-arrest was suggested by onset of expression at the time of epithelial differentiation (approximately embryonic day 13) (32,51), and by similarity within the zinc finger domain to family members EKLF and LKLF that were previously associated with growth-arrest or differentiation-specific gene expression (52,53). Similarity to these other genes is limited to the DNA binding zinc finger region.

The results show that GKLF can induce proliferation when over-expressed in vitro. Analysis of expression in dysplastic cells and tumor cells in vivo provides independent evidence that GKLF exhibits properties expected of an oncogene. Genetic progression of carcinoma appears to involve genes and pathways important for homeostasis of normal epithelium (6,7,9,54). For example, the zinc finger protein GLI is expressed in normal hair shaft keratinocytes, while c-MYC is expressed in normal epithelium of the colonic mucosa. In tumors derived from these tissues, GLI and c-MYC are more frequently activated by recessive genetic changes in upstream components of their respective biochemical pathways than by gain-of-function alterations such as gene amplification. Up-regulation of GKLF expression in dysplastic epithelium and tumor cells in vivo is particularly interesting as expression appears not to be increased by proliferation in vitro. Expression of the endogenous GKLF mRNA in RK3E cells was similar in cycling vs. contact-inhibited cells (data not shown). In contrast, GKLF is significantly induced in NIH3T3 cells during growth-arrest (30). These different results suggest that cell type-specific mechanisms can regulate GKLF expression, and that GKLF may play different roles in epithelial vs. mesenchymal cells.

Squamous epithelium is divided into compartments (55,56). In the basal layer, proliferative stem cells possess unlimited self-renewal capacity, while transit amplifying cells undergo several rounds of mitosis and then withdraw from the cell cycle and terminally differentiate. Proliferation and differentiation are normally balanced such that overall cell number remains constant. In contrast to GLI and c-MYC, GKLF expression in skin appears limited to the differentiating compartment (32). A simple model is that GKLF normally regulates the rate of maturation and shedding and the overall transit time for individual cells. The thickness of epithelium, which varies greatly in development and in different adult tissues, may be regulated not only by alterations in the rate of cell division in the basal layer, but also in response to GKLF or similarly acting molecules in the suprabasal layers. This model is consistent with the relatively late induction of GKLF during mouse development, and is testable by modulating expression of GKLF in transgenic animals or using raft epithelial cultures in vitro. Activation of GKLF in the basal layer of dysplastic epithelium suggests that dysplasia and progression to invasion and metastasis could result from loss of normal compartment-specific patterns of gene expression.

GKLF, c-MYC and GLI are potent oncogenes in epithelioid RK3E cells in vitro, are analogous with respect to their expression in normal epithelium, and have potentially complex roles in the regulation of epithelial cell proliferation, differentiation, or apoptosis (6,7,9,44,56-58). Analysis of well-characterized tumor types such as colorectal carcinoma and basal cell carcinoma of the skin suggests that genetic alterations cluster within specific pathways, rather than within any specific gene, and that these pathways can function as regulators of oncogene transcription (70,71). An activity common to several oncogenes implicated in carcinoma is the ability to induce transformed foci in the RK3E assay (17,72). This assay is highly specific, as foci result from expression of tumor-derived mutant (but not wild-type) alleles of RAS or β-catenin (72), and only GKLF and c-MYC were identified in a large screen. The assay also detects a distinct subset of oncogenies compared with other host cell lines. With the exception of RAS, the oncogenes that transform RK3E cells do not induce foci in NIH3T3 cells.

GKLF encodes a zinc finger transcription factor of the GLI-Krüppel family (73) and is distinct from many other oncogenes in that expression in normal tissue is observed in terminally differentiating epithelial cells. In addition, expression is induced in association with cell growth-arrest in vitro (30). As predicted by these observations, expression in certain tumor-types is reduced compared with the relevant normal epithelia. Thus, GKLF expression is reduced in colorectal tumors, a result supported by multiple approaches including analysis of RNA extracted from tissues (51), SAGE (66), and immunohistochemical analysis of human tissues. In situ hybridization analysis of several prostatic tumors likewise indicates that GKLF is expressed in normal prostatic epithelium, and that expression can be lost during tumor progression.

In contrast to colorectal and prostatic carcinoma, GKLF expression is activated in both invasive carcinoma and preinvasive neoplastic lesions during progression of most breast carcinomas and virtually all oropharyngeal squamous cell carcinomas. Breast and oral cancer share a number of additional molecular alterations. Loss-of-function mutations frequently affect p53 and p16/CDKN2, while a smaller proportion of tumors (5-20%) exhibit gene amplification of c-MYC, cyclin D1, erbB-family members including the EGF receptor and erbB-2/HER-2/neu, or others (74-78). Unlike carcinomas of the GI tract or skin, neither breast nor oral carcinoma is reported to exhibit frequent genetic alterations that activate known transforming oncogenes such as RAS, β-catenin, c-MYC, or GLI. By analogy with oncogenes in other tumor types, disruption of the pathways that control GKLF mRNA expression in breast epithelial cells and in oral mucosa represents a potential mechanism of tumor initiation or progression in vivo.

The pattern of GKLF expression in normal epithelia may provide clues as to how GKLF functions in tumor progression. Stratified squamous epithelium contains at least four functionally-distinct compartments (55,79). The stem cell compartment is composed of cells within the basal cell layer that exhibit a capacity for self-renewal, but which rarely divide. The transit amplifying compartment is composed of cells within the basal or parabasal cell layers that exhibit rapid cell division, but a reduced capacity for self-renewal. Differentiation occurs within the prickle cell layer that contains identifiable desmosomes, leading to the outermost, keratinized superficial layer. While mechanisms regulating transitions from one compartment to the next remain poorly understood, c-MYC activation can induce stem cells to enter the highly proliferative transit amplifying compartment (56). Since self-renewal and rapid cell division occur in distinct cell-types, the organization of compartments enables rapid turnover of epithelial cells while minimizing the possibility of sustaining permanent genetic damage in stem cells.

The observation that GKLF functions normally in the prickle cell layer suggests that each of the three compartments—stem cell, transit amplifying, and prickle layer—expresses a transforming activity or a critical function (e.g., self-renewal or proliferation) that may contribute to progression of carcinoma. These compartments appear to be intermingled in dysplastic stratified squamous epithelium, with prickle layer markers including GKLF misexpressed in the basal layers, while other basal or parabasal markers are misexpressed in superficial layers. Loss of these compartment-specific patterns of gene expression may result in co-expression of properties of several compartments in a single cell. For example, specific properties of the prickle cell layer, such as reduced cellular adhesion to basement membranes, altered adhesion to other cells, and/or loss of the cellular mechanisms that mediate contact inhibition could confer invasive or metastatic properties to oral carcinomas. Although breast epithelium is derived from skin during embryogenesis, the biology and organization of normal breast epithelium is distinguished from skin in many aspects. However, the organization of compartments is likely to be similar, and loss of such organization as a consequence of GKLF activation and other alterations may contribute to tumor progression.

To better understand the mechanism of transformation, transcriptional alterations induced by GKLF are being characterized when expressed in epithelial cells in vitro. In the future, identification of upstream regulators of GKLF transcription in epithelial cells may elucidate the pathways that regulate GKLF, and the mechanism of deregulation of GKLF in specific tumor-types.

### EXAMPLE 24

Subcellular Localization of KFL4/GKLF Identifies Breast Cancer Patients with a Distinct Clinical Outcome

KLF4 encodes a zinc finger transcription factor that was identified as an oncogene using expression cloning in the RK3E epithelial model. Mouse knockout studies revealed an essential role for KLF4 in skin differentiation, consistent with expression of KLF4 in superficial, nondividing cell layers in normal skin and oral mucosa. KLF4 mRNA and protein expression are upregulated at an early step during progression of most breast and oral cancers, but not in colorectal or prostatic carcinoma. Thus, de novo expression of KLF4 within proliferating epithelial compartments may represent a mechanism of tumor initiation or progression.

Ki67, a 395-kd gene product, is a popular marker of cell proliferation in normal and neoplastic tissues associated with the cell cycle. Expression of Ki67 is closely associated with the proliferation phase and is absent during the resting phase of cell cycle5.6. Expression of KLF4 and Ki67 were examined by immunohistochemical staining of normal breast tissue obtained by reduction mammoplasty.

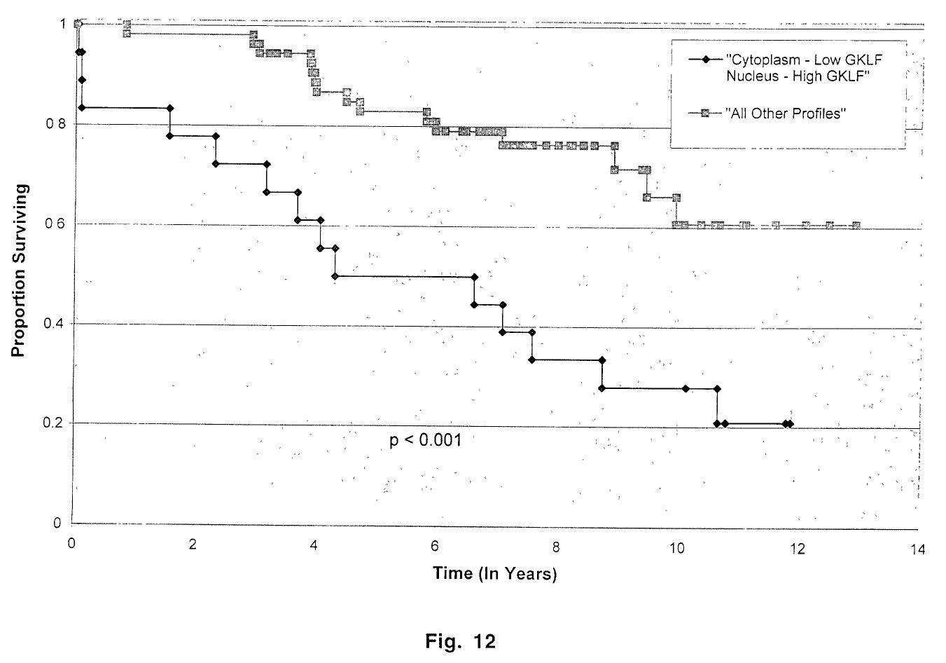

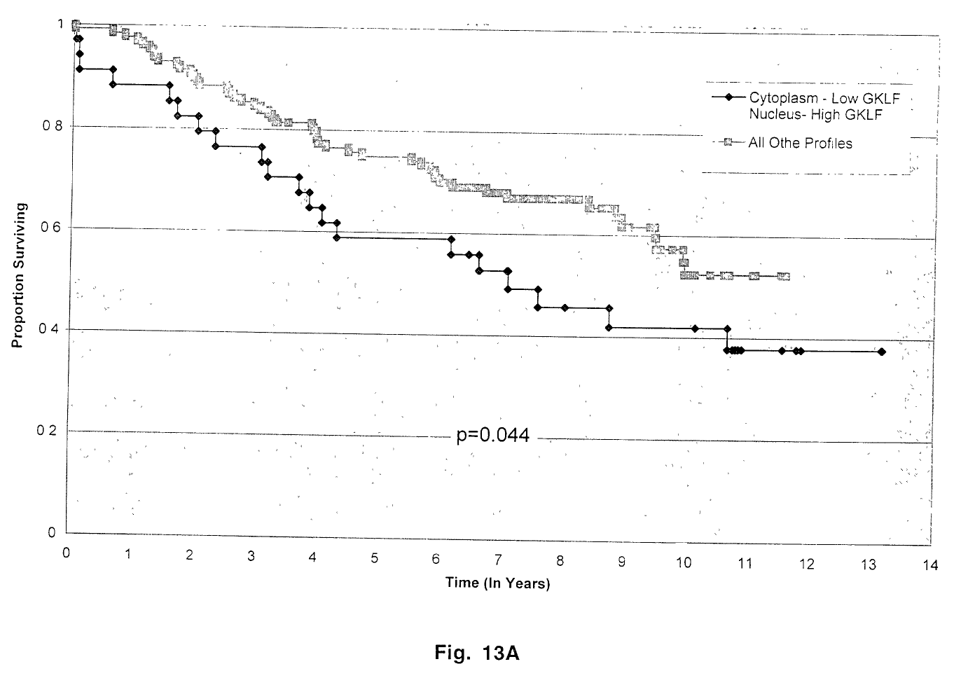

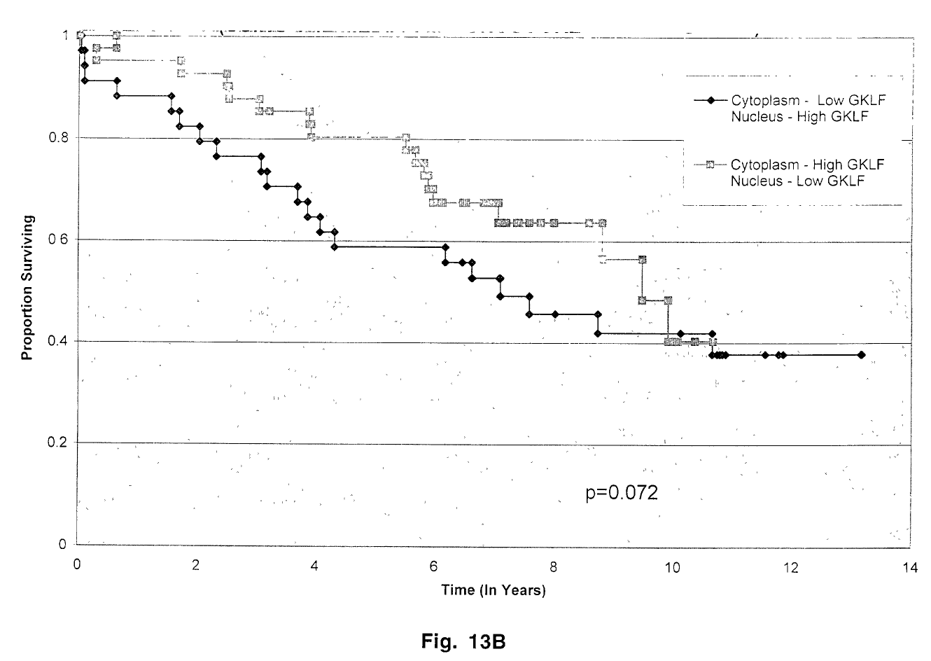

Overall expression of KLF4 is low or undetectable in normal breast epithelium, with a mixed nuclear and cytoplasmic staining pattern. See FIGS. 12, 13A and 13B and Tables 7 and 8. A subset of lobular units exhibit prominent nuclear staining, and these lobules were low or negative for expression of Ki67.

TABLE 7
Characteristics of the Study Population
According to GKLF Cytoplasmic
and Nucleic Staining Profile (Low Cytoplasmic
GKLF and High Nucleic GKI
versus High Cytoplasmic
GKLF and Low Nucleic GKLF)

| | Low Cytoplasmic GK | | High Cytoplasmic GKI | | |
|---|---|---|---|---|---|
| | High Nucleic GKLF | | Low Nucleic GKLF | | |
| | n | % | n | % | P-value |
| Race | | | | | |
| White | 26 | 74.29 | 29 | 70.73 | 0.732 |
| Black | 9 | 25.71 | 12 | 29.27 | |
| Menopausal | | | | | |
| Status | | | | | |
| Pre | 17 | 48.57 | 17 | 41.46 | 0.537 |
| Post | 18 | 51.43 | 24 | 58.54 | |
| Stage | | | | | |
| I | 26 | 78.79 | 23 | 58.97 | 0.074 |
| >I | 7 | 21.21 | 16 | 41.03 | |
| Lymph | | | | | |
| Node | | | | | |
| Negative | 21 | 60.0 | 18 | 51.43 | 0.474 |
| Positive | 14 | 40.0 | 17 | 48.57 | |
| Tumor Size | | | | | |
| ≦2 cm. | 18 | 51.43 | 22 | 53.66 | 0.847 |
| >2 cm. | 17 | 48.57 | 19 | 46.34 | |
| Histologic | | | | | |
| G | | | | | |
| Low | 13 | 38.24 | 15 | 51.72 | 0.287 |
| High | 21 | 61.76 | 14 | 48.28 | |

TABLE 8
Characteristics of the Study Population
According to GKLF Cytoplasmic and
Nucleic Staining Profile (Low
Cytoplasmic and High Nucleic GKLF vs. All
Others)

| | Low Cytoplasmic GKI | | | | |
|---|---|---|---|---|---|
| | High Nucleic GKLF | | All Other Profiles | | |
| | (N = 36) | | (N = 138) | | |
| | n | % | n | % | P-value |
| Race | | | | | |
| White | 26 | 74.29 | 93 | 71.54 | 0.748 |
| Black | 9 | 25.71 | 37 | 28.46 | |
| Menopausal | | | | | |
| Status | | | | | |
| | 17 | 48.57 | 52 | 39.69 | 0.345 |
| Post | 18 | 51.43 | 79 | 60.31 | |
| Stage | | | | | |
| I | 26 | 78.79 | 88 | 68.22 | 0.237 |
| >I | 7 | 21.21 | 41 | 31.78 | |
| Lymph | | | | | |
| Node | | | | | |
| Negative | 21 | 60.0 | 76 | 61.79 | 0.848 |
| Positive | 14 | 40.0 | 47 | 38.21 | |
| Tumor Size | | | | | |
| ≦2 cm. | 18 | 51.43 | 55 | 44.35 | 0.460 |
| >2 cm. | 17 | 48.57 | 69 | 55.65 | |
| Histologic | | | | | |
| G | | | | | |
| Low | 13 | 38.24 | 53 | 54.08 | 0.113 |
| High | 21 | 61.76 | 45 | 45.92 | |

These results indicate that KLF4 may play a normal role in differentiating lobules, consistent with its role in other epithelial tissues such as the skin or the colorectal mucosa. In addition, co-expression of KLF4 and Ki67 may be specific to malignant cells and may help to discriminate between normal breast epithelial cells and malignant cells in clinical samples.

KLF4 expression in breast tumors identifies three distinct patterns: predominantly cytoplasmic, predominantly nuclear, or mixed, with the mixed staining pattern being most common. Initial outcome analysis indicates a 5-year survival rate of 76% for patients with prominent cytosolic staining (52 of 68 patients with>median cytosolic staining survived for 5 years or greater) vs. 60% for patients with low cytosolic staining (38 of 63 patients with<median cytosolic staining; p=0.0464). These results are consistent with a function of nuclear KLF4 as a transforming oncogene, and indicate that activity of the protein is likely to be regulated by subcellular localization in breast tissues. Current studies are aimed at determining Survival rates in groups with distinct nuclear/cytosolic ratios of KLF4, and understanding the mechanisms that regulate subcellular localization in cultured breast tumor cell lines.

The following references were cited herewith.

1. Weinberg, Cancer Research 49: 3713-3721, 1989.

2. Hunter, Cell 64: 249-270, 1991.

3. Bishop, Cell 64: 235-248, 1991.

4. Miki, et al., Methods in Enzymology 254: 196-206, 1995.

5. Look, Science 278: 1059-1064, 1997.

6. He, et al., Science 281: 1509-1512, 1998.

7. Korinek, et al., Nature Genetics 19. 379-383, 1998.

8. Goodrich, et al., Science 277: 1109-1113, 1997.

9. Dahmane, et al., Nature 389: 876-881, 1997.

10. Hahn, et al., Nature Medicine 4: 619-622, 1998.

11. Xu, et al., Cell 62: 599-608, 1990.

12. Chellappan, et al., Cell 65: 1053-1061, 1991.

13. Kallioniemi, et al., Seminars in Cancer Biology 4: 41-46, 1993.

14. Iftner, et al., Journal of Virology 62: 3655-3661, 1988.

15. Lugo, et al., Molecular and Cellular Biology 9: 1263-1270, 1989.

16. Pace, et al., Proc. Natl. Acad. Sci. USA 88: 7031-7035, 1991.

17. Ruppert, et al., Mol. Cell Biol. 11: 1724-1728, 1991.

18. Capobianco, et al., Mol. Cell. Biol. 17: 6265-6273, 1997.

19. Draetta, et al., Current Opinion in Cell Biology 6: 842-846, 1994.

20. Hussussian, et al., Nature Genetics 8: 15-21, 1994.

21. Sherr, et al., Genes & Development 9: 1149-1163, 1995.

22. Weinberg, Cell 81: 323-330, 1995.

23. Bishop, Cell 42: 23-38, 1985.

24. Harlow, et al., Cancer Surveys 12: 161-195, 1992.

25. Nevins et al., Current Topics in Microbiology and Immunology 199. 25-32, 1995.

26. Mal, et al., Nature 380: 262-265, 1996.

27. Whitehead, et al., Mol. Cell. Biol. 15: 704-710, 1995.

28. Frisch, Journal of Cell Biology 127: 1085-1096, 1994.

29. Pear, et al., Proc. Natl. Acad. Sci. USA 90: 8392-8396, 1993.

30. Shields et al., Journal of Biol. Chemistry 271: 20009-20017, 1996.

31. Yet, et al., Journal of Biological Chemistry 273: 1026-1031, 1998.

32. Garrett-Sinha, et al., Jour. of Biol. Chem. 271: 31384-31390, 1996.

33. Littlewood, et al., Nucleic Acids Research 23: 1686-1690, 1995.

34. Merritt, et al., Arc. of Otolaryngology—Head & Neck Surgery 116. 1394-1398, 1990.

35. Leonard et al., International Journal of Cancer 48: 511-515, 1991.

36. Garte, Critical Reviews in Oncogenesis 4: 435-449, 1993.

37. Fracchiolla, et al., Cancer 75: 1292-1301, 1995.

38. Courjal, et al., Cancer Research 57: 4360-4367, 1997.

39. Field, et al., Oncogene 4: 1463-1468, 1989.

40. Eversole, et al., European Journal of Cancer 131-135, 1994.

41. Porter, et al., Acta Oto-Laryngologica 114. 105-109, 1994.

42. Bos, Cancer Research 49: 4682-4689, 1989.

43. Grandori, et al., Trends in Biochemical Sci. 22: 177-181, 1997.

44. Shim et al., Current Topics in Microbiology & Immunology 224: 181-90: 3, 1997.

45. Fischer, et al., Cell Growth & Differentiation 9: 905-918, 1998.

46. Goudie, et al., Nature Genetics 3: 165-169, 1993.

47. Richards, et al., Human Genetics 101: 317-322, 1997.

48. Shields, et al., Nucleic Acids Research 26: 796-802, 1998.

49. Shields, et al., Journal of Biol. Chem. 272: 18504-18507, 1997.

50. Jenkins, et al., Journal of Biol. Chem. 273. 10747-10754, 1998.

51. Tonthat, et al., FEBS Letters 419: 239-243, 1997.

52. Miller, et al., Mol. Cell. Biol. 13: 2776-2786, 1993.

53. Kuo, et al., Science 277: 1986-1990, 1997.

54. Johnson, et al., Science 272: 1668-1671, 1996.

55. Fuchs, et al., Curr. Opinion in Genetics & Devel. 4: 725-736, 1994.

56. Gandarillas, et al., Genes & Development 11: 2869-2882, 1997.

57. Hueber, et al., Science 278: 1305-1309, 1997.

58. Brewster, et al., Nature 393: 579-583, 1998.

59. Chomczynski, et al., Analytical Biochemistry 162: 156-159, 1987.

60. Cheng, et al., Genes & Development 9: 2335-2349, 1995.

61. Nijjar, et al., Cancer Research 59: 5112-5118, 1999.

62. Birkedal-Hansen, et al., Biochemistry 27: 6751-6758, 1988.

63. Grizzle, et al., In: Margaret Hanausek and Zbigniew Walaszek (ecls.), John Walker's Methods in Molecular Medicine—Tumor marker protocols, pp. 161-179. Totowa, N.J.: Humana Press, Inc., 1998.

64. Grizzle, et al., In: Margaret Hanausek and Zbigniew Walaszek (eds.), John Walker's Methods in Molecular Medicine—Tumor Marker Protocols, pp. 143-160. Totowa, N.J.: Humana Press, Inc., 1998.

65. Snedecor, et al., Statistical methods. Ames, Iowa: Iowa State University Press, 1980.

66. Lal, et al., Cancer Research 59: 5403-5407, 1999.

67. Velculescu, et al., Nature Genetics 23: 387-388, 1999.

68. Segre, et al., Nature Genetics 22: 356-360, 1999.

69. Alford, et al., Biochemical Society Symposia 63: 245-259, 1998.

70. Sparks, et al., Cancer Research 58: 1130-1134, 1998.

71. Xie, et al., Nature 391: 90-92, 1998.

72. Kolligs, et al., Mol. Cell. Biol. 19: 5696-5706, 1999.

73. Ruppert, et al., Mol. Cell Biol. 8: 3104-3113, 1988.

74. Cairns, et al., Nat. Genet. 11: 210-212, 1995.

75. Reed, et al., Cancer Research 56: 3630-3633, 1996.

76. Ruppert, et al., Breast Cancer Res & Treatment 44: 93-114, 1997.

77. Ingvarsson, S. Seminars in Cancer Biology 9: 277-288, 1999.

78. Nass, et al., Hematology—Oncology Clinics of North America 13: 311-332, 1999.

79. Watt, F. M. Philosophical Transactions of the Royal Society of London—Series B: Biological Sciences 353: 831-837, 1998.

Any patents or publications mentioned in this specification are indicative of the levels of those skilled in the art to which the invention pertains. Further, these patents and publications are incorporated by reference herein to the same extent as if each individual publication was specifically and individually indicated to be incorporated by reference.

One skilled in the art will appreciate readily that the present invention is well adapted to carry out the objects and obtain the ends and advantages mentioned, as well as those objects, ends and advantages inherent herein. The present examples, along with the methods, procedures, treatments, molecules, and specific compounds described herein are presently representative of preferred embodiments, are exemplary, and are not intended as limitations on the scope of the invention. Changes therein and other uses will occur to those skilled in the art which are encompassed within the spirit of the invention as defined by the scope of the claims.

What is claimed is:

1. A method of detecting transforming activities of a carcinoma oncogene, comprising the steps of:
transforming epithelioid cells with said oncogene; and
detecting morphological transformation, wherein the presence of transformed cell lines indicates that said oncogene has transforming activities.

2. The method of claim 1, wherein said epithelioid cells are RK3E cells.

3. The method of claim 1, wherein said oncogene is selected from the group consisting of RAS, GKLF, c-MYC and GLI.

4. The method of claim 1, wherein said method detects protein coding region of said oncogene without truncation or rearrangement.

5. A method of identifying oncogenicity of a gene, comprising the steps of:
transforming epithelioid cells with said gene;
detecting transformed cell lines; and
measuring tumorigenicity of said transformed cell lines by injecting an animal with said transformed cell lines, wherein induction of tumors in said animal indicates oncogenicity of said gene.

6. The method of claim 5, wherein said epithelioid cells are RK3E cells.

7. A method of identifying oncogene-specificity of a known drug, comprising the steps of:
transforming epithelioid cells with said oncogene;
detecting transformed cell lines; and
contacting said transformed cell lines with said drug, wherein if said drug inhibits proliferation or survival of said transformed cell lines, said drug is specific for inhibiting said oncogene.

8. The method of claim 7, wherein said epithelioid cells are RK3E cells.

9. The method of claim 7, wherein said oncogene is a carcinoma oncogene.

10. The method of claim 9, wherein said oncogene is selected from the group consisting of RAS, GKLF, c-MYC and GLI.

11. A method of screening for a drug functioning as an inhibitor of an oncogene, comprising the steps of:
transforming epithelioid cells with said oncogene;
contacting said cells with said drug; and
detecting transformed cell lines; wherein absence of transformed cell lines or reduced transformed cell lines compared to those obtained without drug contact indicates that said drug is an inhibitor of said oncogene.

12. The method of claim 11, wherein said epithelioid cells are RK3E cells.

13. The method of claim 11, wherein said oncogene is a carcinoma oncogene.

14. The method of claim 13, wherein said oncogene is selected from the group consisting of RAS, GKLF, c-MYC and GLI.

15. A method of screening for alterations in enzyme activity, protein expression, or mRNA expression in association with an oncogene, comprising the steps of:
transforming epithelioid cells with said oncogene; and
measuring the activity or expression level of said enzyme, protein or mRNA, wherein if the activity or expression level of said enzyme, protein or mRNA in transformed cell lines differs from that in non-transformed cell lines, said oncogene regulates said enzyme activity, protein expression, or mRNA expression.

16. The method of claim 15, wherein said epithelioid cells are RK3E cells.

17. The method of claim 15, wherein said oncogene is a carcinoma oncogene.

18. The method of claim 17, wherein said oncogene is selected from the group consisting of RAS, GKLF, c-MYC and GLI.

19. A method of treating an individual having a carcinoma, comprising the step of:
administering a drug to said individual, wherein said drug inhibits the expression/activity of GKLF.

20. The method of claim 19, wherein said carcinoma is selected from the group consisting of breast carcinoma and oral squamous cell carcinoma.

21. A method of monitoring a treatment thereby evaluating effectiveness of the treatment in an individual, comprising the step of:
detecting the expression levels of GKLF in said individual prior to, during and post said treatment, wherein decreases of said expression levels of GKLF indicate effective response of said individual to said treatment, therefore, said treatment is monitored and the effectiveness of said treatment is evaluated in said individual.

22. The method of claim 21, wherein said treatment is selected from the group consisting of drug administration, radiation therapy, gene therapy and chemotherapy.

23. The method of claim 21, wherein said individual suffers from a carcinoma selected from the group consisting of breast carcinoma and oral squamous cell carcinoma.

24. A monoclonal antibody directed against GKLF protein, wherein said antibody is an IgG₁ antibody.

25. A method of monitoring a treatment thereby evaluating effectiveness of the treatment in an individual, comprising the step of:
administering the monoclonal antibody of claim 24 to said individual prior to, during and post said treatment, wherein said antibody detects the localization and level of GKLF protein, and wherein decreases of GKLF protein level indicate effective response of said individual to said treatment, so treatment is monitored and the effectiveness of said treatment is evaluated in said individual.

26. The method of claim 25, wherein said treatment is selected from the group consisting of drug administration, radiation therapy, gene therapy and chemotherapy.

27. The method of claim 25, wherein said individual suffers from a carcinoma selected from the group consisting of breast carcinoma and oral squamous cell carcinoma.

28. A kit for monitoring a treatment thereby evaluating effectiveness of the treatment in an individual, comprising:
the monoclonal antibody of claim 24; and
a suitable carrier.

29. A DNA fragment encoding a Gut-Enriched Krüppel-Like Factor/Epithelial Zinc Finger (GKLF) protein selected from the group consisting of:
(a) isolated DNA which encodes a GKLF protein;
(b) isolated DNA which hybridizes to isolated DNA of (a) above and which encodes a GKLF protein; and
(c) isolated DNA differing from the isolated DNAs of (a) and (b) above in codon sequence due to the degeneracy of the genetic code, and which encodes a GKLF protein.

30. The DNA fragment of claim 29, wherein said DNA has the sequence shown in SEQ ID No: 5.

31. The DNA fragment of claim 29, wherein said GKLF protein has the amino acid sequence shown in SEQ ID No: 6.

32. A vector capable of expressing the DNA fragment of claim 29 adapted for expression in a recombinant cell and regulatory elements necessary for expression of the DNA fragment in the cell.

33. The vector of claim 32, wherein said DNA fragment encodes a GKLF protein having the amino acid sequence shown in SEQ ID No: 6.

34. A host cell transfected with the vector of claim 32, said vector expressing a GKLF protein.

35. The host cell of claim 34, wherein said cell is selected from group consisting of bacterial cells, mammalian cells, plant cells and insect cells.

36. The host cell of claim 35, wherein said bacterial cell is E. coli.

37. Isolated and purified GKLF protein coded for by DNA fragment selected from the group consisting of:
(a) isolated DNA which encodes a GKLF protein;
(b) isolated DNA which hybridizes to isolated DNA of (a) above and which encodes a GKLF protein; and
(c) isolated DNA differing from the isolated DNAs of (a) and (b) above in codon sequence due to the degeneracy of the genetic code, and which encodes a GKLF protein.

38. The isolated and purified GKLF protein of claim 37 having the amino acid sequence shown in SEQ ID No: 6.

39. A method of identifying the prognosis of an individual thereby allowing selection of a more effective, less invasive or a less toxic therapeutic alternative to individual patients having a breast tumor, comprising the step of:
examining the expression of KLF4 in said breast tumor.

40. The method of claim 39, wherein said expression is examined using immunohistochemistry.

41. The method of claim 39, wherein said immunohistochemistry employs a monoclonal antibody directed against KLF4 protein.

42. The method of claim 39, wherein a predominantly cytosolic staining indicates a greater likelihood of survival of the individual or a greater likelihood of response to a specific therapy.

43. The method of claim 39, wherein a predominantly nuclear staining and a lower cytosolic staining indicates a lower likelihood of survival of the individual or a lower likelihood of response to a specific therapy.

44. The method of claim 39, wherein said tumor is smaller than about 2 cm.

45. A cell line generating a monoclonal antibody directed against KLF4 protein.

46. The monoclonal antibody of claim 24, wherein said antibody is designated IE5/IE2.
